SEQUENCE LISTING (9 sequences)

SEQ ID NO: 1 - DNA, 1382 bp, Homo sapiens
cggatgaaggctatttgaagtcgccataacctggtcagaagtgtgcctgtcggcggggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgattccagaatttttctgtgatacatgaagactttccatatcaagagacatggtattgactcaacagtttccagtcatggccaaatgttcaatatgagtctcaataaactgaatttttcttgcgaaaaaaaaaaaaaaaaaaaaaaaaaaaaaaaaaaaaaaaaaaaaaaaaaa

SEQ ID NO: 2 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 3 - DNA, 3604 bp, Homo sapiens
gcatctcctcctccctctccccgggctcctactggcctgaggttgagggcggctgggggctcggggcaggctccgcggcgttcccctccccaccccggccctccgttcagccgcgctcctccggggctgcggttcctactgcgcgagctgccagtggattcgctcttttcctccgtccgtggcccgcctgggcggccttgttctttccgcagcagccagataaccttctgttcggtgatgaaattatcactaatggttttcattcctgtgaaagtgatgaggaggatagagcctcacatgcaagctctagtgactggactccaaggccacggataggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggaacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaacttaaaaaaaaaaaaaaaaa

SEQ ID NO: 4 - protein, 452 aa, Homo sapiens
MFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 5 - DNA, 1925 bp, Homo sapiens
agtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccattcctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaaccctcggccagcaggtcccctatgccaccaagggcaaccagtgggtaggatacgacgaccaggaaagcgtcaaaagcaaggtgcagtacctgaaggataggcagctggcaggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcacttaggaacgtaatcgtgtcccctatcctacttccccttcctaattccacagctgctcaataaagtacaagagtttaacagtgtgttggcgctttgctttggtctatctttgagcgcccactagacccactggactcacctcccccatctcttctgggttccttcctctgagccttgggacccctgagcttgcagagatgaaggccgccatgtt

SEQ ID NO: 6 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRTLGQQVPYATKGNQWVGYDDQESVKSKVQYLKDRQLAGAMVWALDLDDFQGSFCGQDLRFPLTNAIKDALAAT

SEQ ID NO: 7 - DNA, 1867 bp, Homo sapiens
cacatagctcagttcccataaaagggctggtttgccgcgtcggggagtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtgaaaaaaaaaaaaaaaaaaaaaaaaaaaaaa

SEQ ID NO: 8 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 9 - DNA, 1376 bp, Homo sapiens
gtaagaacactctcgtgagtctaacggtcttccggatgaaggctatttgaagtcgccataacctggtcagaagtgtgcctgtcggcggggagagaggcaatatcaaggttttaaatctcggagaaatggctttcgtttgcttggctatcggatgcttatatacctttctgataagcacaacatttggctgtacttcatcttcagacaccgagataaaagttaaccctcctcaggattttgagatagtggatcccggatacttaggttatctctatttgcaatggcaacccccactgtctctggatcattttaaggaatgcacagtggaatatgaactaaaataccgaaacattggtagtgaaacatggaagaccatcattactaagaatctacattacaaagatgggtttgatcttaacaagggcattgaagcgaagatacacacgcttttaccatggcaatgcacaaatggatcagaagttcaaagttcctgggcagaaactacttattggatatcaccacaaggaattccagaaactaaagttcaggatatggattgcgtatattacaattggcaatatttactctgttcttggaaacctggcataggtgtacttcttgataccaattacaacttgttttactggtatgagggcttggatcatgcattacagtgtgttgattacatcaaggctgatggacaaaatataggatgcagatttccctatttggaggcatcagactataaagatttctatatttgtgttaatggatcatcagagaacaagcctatcagatccagttatttcacttttcagcttcaaaatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgattccagaatttttctgtgatacatgaagactttccatatcaagagacatggtattgactcaacagtttccagtcatggccaaatgttcaatatgagtctcaataaactgaatttttcttgcgaatgttgaaaaa

(19) United States
(12) Patent Application Publication
(10) Pub. No.: US 2016/0166520 A1
(43) Pub. Date: Jun. 16, 2016

(54) METHODS AND COMPOSITIONS RELATING TO THE TREATMENT OF VISCERAL FAT AND ASTHMA

(71) Applicants: YALE UNIVERSITY, New Haven, CT (US); BROWN UNIVERSITY, Providence, RI (US)

(72) Inventors: Jack A. ELIAS, Providence, RI (US); Farida AHANGARI, Madison, CT (US); Chun Geun LEE, Woodbridge, CT (US)

(73) Assignees: BROWN UNIVERSITY, Providence, RI (US); YALE UNIVERSITY, New Haven, CT (US)

(21) Appl. No.: 14/969,140

(22) Filed: Dec. 15, 2015

Related U.S. Application Data
(60) Provisional application No. 62/091,814, filed on Dec. 15, 2014.

(51) Int. Cl. A61K 31/166; C12Q 1/68

(52) U.S. Cl. CPC A61K 31/166; C12Q 1/6883; C12Q 2600/158; C12Q 2600/112; C12Q 2600/106

(57) ABSTRACT

The technology described herein is directed to methods of treating, e.g., obesity and/or asthma which relate to the interacting roles of Chi3L1 and Sirt1 in controlling adipose tissue and respiratory health.

### CROSS-REFERENCE TO RELATED APPLICATIONS

This application claims benefit under 35 U.S.C. §119(e) of U.S. Provisional Application No. 62/091,814 filed Dec. 15, 2014, the contents of which are incorporated herein by reference in their entirety.

### GOVERNMENT SUPPORT

This invention was made with government support under Grant Nos. UO1-HL108638, RO1-093017, UH2-HL123876, RO1-HL115813, K23-HL094531 awarded by the National Institutes of Health. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Dec. 9, 2015, is named 058040-082981-US_SL.txt and is 28,002 bytes in size.

### TECHNICAL FIELD

The technology described herein relates to the treatment of, e.g., obesity, asthma, and/or obesity-related asthma.

### BACKGROUND

The world is currently experiencing contemporaneous epidemics of obesity and asthma. In the U.S.A., the prevalence of obesity increased from approximately 15% in 1975 to 35% in 2010. Epidemiologic studies have consistently demonstrated that obesity is a risk factor for asthma incidence and prevalence, as well as increased asthma severity and poor disease control. Overall, the association between obesity and asthma is so striking that obese subjects with asthma are now considered to represent a ‘unique’ asthma phenotype that does not respond well to inhaled corticosteroids. However, the mechanisms that link obesity and asthma remain poorly understood.

### SUMMARY

The inventors have discovered that both a high-fat diet and allergen challenge cause increases in the expression of Chi3L1. Furthermore, Chi3l1 itself contributes to fat accumulation and inflammation in the lung. Finally, it was found that the level of Chi3L1 controls the role of Sirt1 in obesity and asthma—as Chi3L1 increases, Sirt1 transitions from a protective role into actually contributing to disease development. Accordingly, described herein are treatments for, e.g., obesity and asthma that have been developed from this new understanding of the interplay of Chi3L1, Sirt1, obesity, and asthma.

In one aspect of any of the embodiments, described herein is a method of treating obesity, visceral fat accumulation, asthma or obesity-related asthma, the method comprising administering an inhibitor of Chi3L1 to a subject in need of treatment for obesity, visceral fat accumulation, asthma or obesity-related asthma. In one aspect of any of the embodiments, described herein is a method of treating asthma, the method comprising administering an inhibitor of Chi3L1 to a subject in need of treatment for asthma. In some embodiments of any of the aspects, the subject is obese or diagnosed as having obesity-related asthma. In some embodiments of any of the aspects, the method further comprises administering a Sirt1 inhibitor.

In one aspect of any of the embodiments, described herein is a method of treating obesity, visceral fat accumulation, asthma or obesity-related asthma in a subject in need thereof, the method comprising administering a Sirt1 agonist to a subject determined to have a decreased level of Chi3L1 expression relative to a reference level.

In some embodiments of any of the aspects, the Chi3L1 inhibitor is targeted or administered to white adipose tissue. In some embodiments of any of the aspects, the Chi3L1 inhibitor is targeted or administered to pulmonary white adipose tissue. In some embodiments of any of the aspects, the Chi3L1 inhibitor is targeted or administered to white adipose tissue. In some embodiments of any of the aspects, the Chi3L1 inhibitor is an antibody, antigen-binding portion thereof, or inhibitory nucleic acid. In some embodiments of any of the aspects, the Chi3L1 inhibitor inhibits a Chi3L1 receptor. In some embodiments of any of the aspects, the Chi3L1 receptor is IL-13 receptor alpha 2 (IL-13Rα2).

In one aspect of any of the embodiments, described herein is a method of treating asthma in a subject in need thereof, the method comprising administering a Sirt1 agonist to a subject who is not obese or not diagnosed as having obesity-related asthma. In some embodiments of any of the aspects, the subject is determined to have a decreased level of Chi3L1 expression relative to a reference level.

In one aspect of any of the embodiments, described herein is a method of treating obesity, visceral fat accumulation, asthma or obesity-related asthma in a subject in need thereof, the method comprising administering a Sirt1 inhibitor to a subject determined to have an increased level of Chi3L1 expression relative to a reference level. In some embodiments of any of the aspects, the subject is not administered a Chi3L11 inhibitor.

In one aspect of any of the embodiments, described herein is a method of treating asthma in a subject in need thereof, the method comprising administering a Sirt1 inhibitor to a subject who is obese or diagnosed as having obesity-related asthma. In some embodiments of any of the aspects, the subject is determined to have an increased level of Chi3L1 expression relative to a reference level. In some embodiments of any of the aspects, the subject is not administered a Chi3L11 inhibitor.

In some embodiments of any of the aspects, the level of Chi3L1 is the level of serum Chi3L1. In some embodiments of any of the aspects, the level of Chi3L1 is the level of sputum Chi3L1.

### BRIEF DESCRIPTION OF THE DRAWINGS

FIGS. 1A-1B demonstrate high fat diet regulation of Chi3l1 gene expression. Wild type (WT) mice received HFD or regular chow (RC). FIG. 1A depicts the levels of mRNA encoding Chi3l1 in epididymal fat pad. FIG. 1B depicts the levels of mRNA encoding Chi3l1 in total lung tissue. The noted values represent the mean±SEM of a minimum of 5 animals. (*p≦0.05, **p≦0.01).

FIGS. 2A-21 demonstrate the role of Chi3l1 on visceral fat accumulation in mice. FIG. 2A depicts a representative photograph showing epididymal fat pads in WT and Chi3l1^{-/-} mice after 12 weeks Regular Diet (RC), FIGS. 2B-2C depict the weight of epidydymal fat pads in WT and Chi3l1^{-/-} mice on RC and a HFD, respectively, FIG. 2D-2E depict the ratio of epidydymal fat pad to the total body weight with RC and HFD, respectively, FIG. 2F depicts the weight of peri-renal fat pads from WT and Chi3l1^{-/-} mice on a HFD, FIG. 2G depicts the ratio of peri-renal fat to the total body weight on HFD, FIG. 2H depicts an H& E photomicrograph of epididymal fat pads from WT and Chi3l1^{-/-} mice. FIG. 2I depicts morphometric evaluation of adipocyte cell size after 12 wks on a HFD. The noted values in FIGS. 2B, 2C 2D-2G and 21 represent the mean±SEM of a minimum of 5 animals. (*p≦0.05, **p≦0.01).

FIGS. 3A-3E demonstrate Chi3l1 regulation of murine epididymal fat pad cytokines. FIGS. 3A-3C depict the levels of mRNA encoding the noted cytokines in fat pads from WT and Chi3l1^{-/-} mice: (FIG. 3A) TNFα, (FIG. 3B) IL-10, (FIG. 3C) IL-6. FIGS. 3D-3E depict the levels of IL-10 released by adipocytes and epididymal fat pad (100 mg), respectively, after 24 hours in culture from WT and chi3l1^{-/-} mice. The noted values represent the mean±SEM of a minimum of 5 animals. (*p≦0.05, **p≦0.01).

FIGS. 4A-4D demonstrate Chi3l1 in murine allergen (OVA)- and HFD-stimulated pulmonary inflammation visceral fat accumulation. WT mice were treated with vehicle (PBS) or sensitized and challenged with aeroallergen (OVA). FIG. 4A depicts the levels of Chi3l1 mRNA in total lung tissue, FIG. 4B depicts Chi3l1 proteins detected by Western blot from total lung tissues, FIG. 4C depicts inflammatory cell (total cell and eosinophil) recovery in BAL from WT and Chi3l1^{-/-} mice challenged with OVA allergen and also fed with regular chow or HFD. FIG. 4D depicts the weight of epididymal fat pad from the mice challenged with OVA allergen and also fed with regular chow or HFD. The values in FIGS. $a, 4C and 4D represent the mean±SEM of a minimum of 5 animals. (*p≦0.05, **P≦0.01, ns, not significant).

FIGS. 5A-5D demonstrate Chi3l1 regulation of Sirt1 gene expression. FIG. 5A depicts Sirt1 mRNA levels in lungs from WT and Chi3l1^{-/-} mice, FIG. 5B depicts Sirt1 mRNA levels in the lungs from WT and Chi3l1^{-/—} mice after OVA sensitization and challenge, FIG. 5C depicts Sirt1 mRNA levels in epididymal fat pads from mice on RC, FIG. 5D depicts Sirt1 mRNA levels in epididymal fat pads from mice on a HFD. The noted values represent the mean±SEM of a minimum of 5 animals. (*P≦0.05, **P≦0.01).

FIGS. 6A-6H demonstrate the effects of the Sirt1 inhibitor (Sirtinol) on lung Th2 inflammation. WT and Chit3l1^{-/-} mice were sensitized and challenged with OVA (ova+) or PBS (ova−) and were treated with Sirtinol or its vehicle (DMSO). Each animal received drug (0.4 mg/kg body weight per day) or vehicle one hour before every antigen challenge. FIGS. 6A-6B depict BAL total cell and eosinophil recovery, FIG. 6C depicts H&E photomicrographs of lungs from WT and Chi3l1^{-/-} mice treated with sirtinol or vehicle after OVA challenge, FIGS. 6D-6H depict the levels of mRNA encoding IL-4, IL-13, IL-17, IL-6 and TNF-α in the lungs of WT and Chi3l1^{-/-} mice treated with Sirtinol or DMSO. The noted values represent the mean±SEM of a minimum of 5 animals. (*p≦0.05, **p≦0.01).

FIGS. 7A-7B demonstrate the effects of Sirt1 inhibition on fat accumulation. WT and Chi3l1^{-/-} mice were treated with Sirtinol or its vehicle (DMSO) and fat accumulation was assessed. Each animal received drug (0.4 mg/kg body weight per day) or vehicle twice per week for 5 weeks. FIG. 7A depicts a representative photograph illustrating abdominal fat accumulation, FIG. 7B depicts the weight of epididymal fat pads from WT and chi3l1^{-/-} mice. The noted values represent the mean±SEM of a minimum of 5 animals. (*P≦0.05, **p≦0.01).

### DETAILED DESCRIPTION

As described herein, increased levels of Chi3L1 contribute to the development of excessive adipose tissue, inflammatory processes, and asthma. Accordingly, in one aspect of any of the embodiments, described herein is a method of treating obesity, visceral fat accumulation, asthma or obesity-related asthma, the method comprising administering an inhibitor of Chi3L1 to a subject in need of treatment for obesity, visceral fat accumulation, asthma or obesity-related asthma.

In one aspect of any of the embodiments, described herein is a method of treating asthma, the method comprising administering an inhibitor of Chi3L1 to a subject in need of treatment for asthma. In some embodiments, subject is obese or diagnosed as having obesity-related asthma.

As used herein, “CHI3L1,” “chintinase-3-like protein 1,” or “YKL-40” refers to a ˜40 kDa glycoprotein secreted by at least macrophages, chondrocytes, neutrophils, synovial cells, and some cancer cells. CHI3L1 does not have chitinase activity, is a Th2 promoting cytokine, has been linked to the AKT anti-apoptotic signaling pathway and induces the migration of astrocytes. The sequences of CHI3L1 expression products are known for a number of species, e.g., human CHI3L1 (NCBI Gene ID No: 1116) mRNA (SEQ ID NO: 5; NCBI Ref Seq: NM_001276.1 and SEQ ID NO: 7; NCBI Ref Seq: NM_001276.2) and polypeptide (SEQ ID NO: 6; NCBI Ref Seq: NP_001267.1 and SEQ ID NO: 8; NCBI Ref Seq: NP_001267.2). The activity of CHI3L1 can be measured, e.g., by measuring the anti-apoptotic effects of CHI3L1, e.g. in response to bleomycin injury, or by measuring the level of CHI3L1-induced fibroproliferation, e.g., in response to bleomycin injury.

As used herein, the term “inhibitor” refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, Chi3L1, e.g. its ability to decrease the level and/or activity of Chi3L1 can be determined, e.g. by measuring the level of an expression product of Chi3L1 and/or the activity of Chi3L1. Methods for measuring the level of a given mRNA and/or polypeptide are known to one of skill in the art, e.g. RT-PCR with primers can be used to determine the level of RNA and Western blotting with an antibody (e.g. an anti-Chi3L1 antibody, e.g. Cat No. 86428; Abcam; Cambridge, Mass.) can be used to determine the level of a polypeptide. The activity of, e.g. Chi3L1 can be determined using methods known in the art and described elsewhere herein. In some embodiments of any of the aspects, the inhibitor can be an inhibitory nucleic acid; an aptamer; an antibody reagent; an antibody; or a small molecule. In some embodiments of any of the aspects, the inhibitor is an antibody, antigen-binding portion thereof, or inhibitory nucleic acid.

In some embodiments of any of the aspects, the inhibitor of Chi3L1 can be an inhibitor of a Chi3L1 receptor. In some embodiments of any of the aspects, the Chi3L1 receptor can be IL-13 receptor alpha 2 (IL-13Rα2). As used herein, “IL-13Rα2,” “Interleukin-13 receptor subunit alpha-2,” or “CD213A2” refers to a membrane bound protein that binds to IL-13 with very high affinity, and negatively regulates both IL-13 and IL-4. IL-13Rα2 competes with the IL-13 receptor comprising IL-13Rα1 and IL4Ralpha for binding of IL-13. Sequences for IL-13Rα2 expression products are known for a number of species, e.g., human IL-13Rα2 (NCBI Gene ID: 3598) mRNA (SEQ ID NO: 1; NCBI Ref Seq: NM_000640.1 and SEQ ID NO: 9; NCBI Ref Seq: NM_000640.2) and polypeptide (SEQ ID NO: 2; NCBI Ref Seq: NP_000631).

In some embodiments of any of the aspects, the inhibitor or agonist administered according to the methods described herein is targeted or administered to white adipose tissue. In some embodiments of any of the aspects, the inhibitor or agonist administered according to the methods described herein is targeted or administered to pulmonary white adipose tissue. In some embodiments of any of the aspects, the inhibitor or agonist administered according to the methods described herein is targeted or administered to white adipose tissue.

As described herein, increased levels of Chi3L1 contribute to pathology, at least in part, by interacting with Sirt1, such that increased levels of Chi3L1 cause Sirt1 to contribute to the development and/or progression of the conditions described herein. Accordingly, when Chi3L1 levels are elevated, Sirt1 inhibitors can be therapeutic, either alone or in combination with a Chi3L1 inhibitor. In some embodiments of any of the aspects, a subject treated with a Chi3L1 inhibitor according to any of the methods described herein can be further administered a Sirt1 inhibitor.

In one aspect of any of the embodiments, described herein is a method of treating obesity, visceral fat accumulation, asthma or obesity-related asthma in a subject in need thereof, the method comprising administering a Sirt1 inhibitor to a subject determined to have an increased level of Chi3L1 expression relative to a reference level. In some embodiments, the subject is not administered a Chi3L11 inhibitor.

In one aspect of any of the embodiments, described herein is a method of treating asthma in a subject in need thereof, the method comprising administering a Sirt1 inhibitor to a subject who is obese or diagnosed as having obesity-related asthma. In some embodiments, the subject is determined to have an increased level of Chi3L1 expression relative to a reference level. In some embodiments, the subject is not administered a Chi3L11 inhibitor. Sirt1 inhibitors are known in the art and can include, by way of non-limiting example, Sirtinol; Tenovin-6; Sirtuin inhibitor IV; (S)-35; EX-527; Sumarin Sodium; Salermide; selisistat; splitomicin; AGK2; and cambinol.

Conversely, when Chi3L1 levels are not elevated, Sirt1 itself can counteract the development and/or progression of the conditions described herein. Accordingly, in one aspect of any of the embodiments, described herein is a method of treating obesity, visceral fat accumulation, asthma or obesity-related asthma in a subject in need thereof, the method comprising administering a Sirt1 agonist to a subject determined to have a decreased level of Chi3L1 expression relative to a reference level.

In one aspect of any of the embodiments, described herein is a method of treating asthma in a subject in need thereof, the method comprising administering a Sirt1 agonist to a subject who is not obese or not diagnosed as having obesity-related asthma. In some embodiments, the subject is determined to have a decreased level of Chi3L1 expression relative to a reference level.

As used herein, “Sirt1” or “sirtuin 1” refers to a class I sirtuin that de-acetylates the PGC1-alpha/ERR-alpha complex. Sequences for Sirt1 expression products are known for a number of species, e.g., human Sirt1 (NCBI Gene ID: 23411) mRNA (SEQ ID NO: 3; NCBI Ref Seq: NM_001142498) and polypeptide (SEQ ID NO: 4; NCBI Ref Seq: NP_001135970).

As used herein, the term “agonist” refers to any agent that increases the level and/or activity of the target, e.g, of Sirt1. As used herein, the term “agonist” refers to an agent which increases the expression and/or activity of the target by at least 10% or more, e.g. by 10% or more, 50% or more, 100% or more, 200% or more, 500% or more, or 1000% or more. Non-limiting examples of agonists of Sirt1 can include Sirt1 polypeptides or fragments thereof and nucleic acids encoding a Sirt1 polypeptide, e.g. a polypeptide comprising the sequence SEQ ID NO: 4 or a nucleic acid comprising the sequence of SEQ ID NO: 3 or variants thereof. Sirt1 agonists are known in the art and can include, by way of non-limting example, resveratrol; resVida; Longevinex; SRT501; SRT1720; SRT2104; and SRT2379.

Levels of CHI3L1, as demonstrated herein, are correlated with obesity-related asthma and indicative of the therapeutic approach that should be taken with modulators of Sirt1. In some embodiments of any of the aspects, the methods and assays described herein include determining the level of Chi3L1 expression. In some embodiments of any of the aspects, the methods and assays described herein include (a) transforming the CHI3L1 into a detectable target; (b) measuring the amount of the target; and (c) comparing the amount of the gene target to an amount of a reference.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, a DNA sample can be digested into fragments by one or more restriction enzyme, or an exogenous molecule can be attached to a fragmented DNA sample with a ligase. In some embodiments, a DNA sample can undergo enzymatic replication, e.g., by polymerase chain reaction (PCR).

Transformation, measurement, and/or detection of a target molecule, e.g. a CHI3L1 mRNA or polypeptide can comprise contacting a sample obtained from a subject with a reagent (e.g. a detection reagent) which is specific for the target, e.g., a CHI3L1-specific reagent. In some embodiments, the target-specific reagent is detectably labeled. In some embodiments, the target-specific reagent is capable of generating a detectable signal. In some embodiments, the target-specific reagent generates a detectable signal when the target molecule is present.

Methods to measure CHI3L1 gene expression products are well known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for CHI3L1 are commercially available and can be used for the purposes of the invention to measure protein expression levels, e.g. anti-CHI3L1 (Cat. No. ab86428; Abcam, Cambridge Mass.). Alternatively, since the amino acid sequences for CHI3L1 are known and publically available at NCBI website, one of skill in the art can raise their own antibodies against these polypeptides of interest for the purpose of the invention.

The amino acid sequences of the polypeptides described herein, e.g. CHI3L1 have been assigned NCBI accession numbers for different species such as human, mouse and rat. In particular, the NCBI accession numbers for the amino acid sequence of human CHI3L1 is included herein, e.g. SEQ ID NO: 6.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiment, the immunoassay can be a quantitative or a semi-quantitative immunoassay.

An immunoassay is a biochemical test that measures the concentration of a substance in a biological sample, typically a fluid sample such as urine, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine, as well as a quality control check in various industries.

In one embodiment, an ELISA involving at least one antibody with specificity for the particular desired antigen (e.g., CHI3L1 as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce much color change Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests are used for medical diagnostics either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick test, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In some embodiments, the level of, e.g., CHI3L1, can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the genes described herein, e.g. CHI3L1. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNA protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein, e.g., CHI3L1, have been assigned NCBI accession numbers for different species such as human, mouse and rat. For example, the human CHI3L1 mRNA (e.g. SEQ ID NO: 5) is known. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i.e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylene diaminetetraacetic acid (EDTA).

A level which is greater than a reference level can be a level which is greater by at least about 10%, at least about 20%, at least about 50%, at least about 100%, at least about 200%, at least about 300%, at least about 500%, at least about 1000%, or greater than the reference level. In some embodiments, a level which is greater than a reference level can be a level which is statistically significantly greater than the reference level. In some embodiments, the reference can be a level of CHI3L1 in a population of subjects who do not have or are not diagnosed as having, and/or do not exhibit signs or symptoms of obesity and/or obesity-related asthma. In some embodiments, the reference can also be a level of expression of CHI3L1 in a control sample, a pooled sample of control individuals or a numeric value or range of values based on the same. In some embodiments, the reference can be the level of CHI3L1 in a sample obtained from the same subject at an earlier point in time, e.g., the methods described herein can be used to determine if a subject's risk or likelihood of developing asthma, e.g., obesity-related asthma is increasing.

A level which is less than a reference level can be a level which is less by at least about 10%, at least about 20%, at least about 50%, at least about 90%, or less than the reference level. In some embodiments, a level which is less than a reference level can be a level which is statistically significantly less than the reference level. In some embodiments, the reference can be a level of CHI3L1 in a population of subjects have or are diagnosed as having, and/or exhibit signs or symptoms of obesity and/or obesity-related asthma. In some embodiments, the reference can also be a level of expression of CHI3L1 in a control sample, a pooled sample of control individuals or a numeric value or range of values based on the same. In some embodiments, the reference can be the level of CHI3L1 in a sample obtained from the same subject at an earlier point in time, e.g., the methods described herein can be used to determine if a subject's risk or likelihood of developing asthma, e.g., obesity-related asthma is increasing.

In some embodiments of any of the aspects, the level of Chi3L1 can be the level of serum Chi3L1. In some embodiments of any of the aspects, the level of Chi3L1 can be the level of sputum Chi3L1. In some embodiments of any of the aspects, the level of Chi3L1 can be the level of pulmonary Chi3L1. In some embodiments of any of the aspects, the level of Chi3L1 can be the level of visceral fat Chi3L1.

In some embodiments, the level of Chi3L1 in a nonobese subject can be from about 5 to about 50 ng/mL. In some embodiments, the level of Chi3L1 in a nonobese subject can be from about 10 to about 40 ng/mL. In some embodiments, the level of Chi3L1 in a nonobese subject can be from 10 to 40 ng/mL. In some embodiments, the level of Chi3L1 in a nonobese subject can be a mean level of about 36 ng/mL. In some embodiments, the level of Chi3L1 in a nonobese subject can be a mean level of 36 ng/mL. In some embodiments, the level of Chi3L1 in an obese subject can be from about 20 to about 400 ng/mL. In some embodiments, the level of Chi3L1 in an obese subject can be from about 30 to about 350 ng/mL. In some embodiments, the level of Chi3L1 in an obese subject can be from 30 to 350 ng/mL. In some embodiments, the level of Chi3L1 in an obese subject can be a mean level of about 65 ng/mL. In some embodiments, the level of Chi3L1 in an obese subject can be a mean level of 65 ng/mL.

In some embodiments, the level of Chi3L1 in an asthmatic subject can be from about 5 to about 80 ng/mL. In some embodiments, the level of Chi3L1 in an asthmatic subject can be from about 10 to about 70 ng/mL. In some embodiments, the level of Chi3L1 in an asthmatic subject can be from 10 to 70 ng/mL. In some embodiments, the level of Chi3L1 in an asthmatic subject can be a mean level of about 65 ng/mL. In some embodiments, the level of Chi3L1 in an asthmatic subject can be a mean level of 65 ng/mL. In some embodiments, the level of Chi3L1 in an obese asthmatic subject can be from about 20 to about 400 ng/mL. In some embodiments, the level of Chi3L1 in an obese asthmatic subject can be from about 30 to about 350 ng/mL. In some embodiments, the level of Chi3L1 in an obese asthmatic subject can be from 30 to 350 ng/mL. In some embodiments, the level of Chi3L1 in an obese asthmatic subject can be a mean level of about 67 ng/mL. In some embodiments, the level of Chi3L1 in an obese asthmatic subject can be a mean level of 67 ng/mL.

In some embodiments, the level of expression products of no more than 200 other genes is determined. In some embodiments, the level of expression products of no more than 100 other genes is determined. In some embodiments, the level of expression products of no more than 20 other genes is determined. In some embodiments, the level of expression products of no more than 10 other genes is determined.

In some embodiments of the foregoing aspects, the expression level of a given gene, e.g., CHI3L1, can be normalized relative to the expression level of one or more reference genes or reference proteins.

The term “sample” or “test sample” as used herein denotes a sample taken or isolated from a biological organism, e.g., a blood or plasma sample from a subject. Exemplary biological samples include, but are not limited to, a biofluid sample; serum; plasma; urine; saliva; and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, the test sample can be a blood sample. In some embodiments, the test sample can be a sputum or serum sample.

The test sample can be obtained by removing a sample from a subject, but can also be accomplished by using previously sample (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments, the methods, assays, and systems described herein can further comprise a step of obtaining a test sample from a subject. In some embodiments, the subject can be a human subject. In some embodiments, the subject can be a subject in need of treatment for (e.g. having or diagnosed as having) asthma, obesity, and/or obesity-related asthma.

The term “obesity” refers to excess fat in the body. Obesity can be determined by any measure accepted and utilized by those of skill in the art. Currently, an accepted measure of obesity is body mass index (BMI), which is a measure of body weight in kilograms relative to the square of height in meters. Generally, for an adult over age 20, a BMI between about 18.5 and 24.9 is considered normal, a BMI between about 25.0 and 29.9 is considered overweight, a BMI at or above about 30.0 is considered obese, and a BMI at or above about 40 is considered morbidly obese. (See, e.g., Gallagher et al. (2000) Am J Clin Nutr 72:694-701.) These BMI ranges are based on the effect of body weight on increased risk for disease. Although BMI correlates with body fat, the relation between BMI and actual body fat differs with age and gender. For example, women are more likely to have a higher percent of body fat than men for the same BMI. Furthermore, the BMI threshold that separates normal, overweight, and obese can vary, e.g. with age, gender, ethnicity, fitness, and body type, amongst other factors. In some embodiments, a subject with obesity can be a subject with a body mass index of at least about 25 kg/m². In some embodiments, a subject with obesity can be a subject with a body mass index of at least about 30 kg/m².

Visceral fat accumulation is the accumulation and/or increase of adipose tissue, e.g., white adipose tissue around internal organs.

Asthma refers to a chronic inflammatory disease of the respiratory system in which the airway occasionally constricts, becomes inflamed, and is lined with excessive amounts of mucus, often in response to one or more triggers. Asthma can be defined simply as reversible airway obstruction in an individual over a period of time. Asthma can be allergic/atopic or non-allergic. Asthma is characterized by the presence of cells such as eosinophils, mast cells, basophils, and activated T lymphocytes in the airway walls. With chronicity of the process, secondary changes occur, such as thickening of basement membranes and fibrosis. The disease is characterized by increased airway hyperresponsiveness to a variety of stimuli, and airway inflammation and constriction. This airway narrowing causes symptoms such as wheezing, shortness of breath, chest tightness, and coughing. The airway constriction responds to bronchodilators. Between episodes, most patients feel well but can have mild symptoms and they can remain short of breath after exercise for longer periods of time than the unaffected individual. The symptoms of asthma can range from mild to life threatening.

Asthma can be triggered by such things as exposure to an allergen (allergic asthma), or non-allergens (non-allergic asthma) such as cold air, pollution (e.g., ozone), warm air, moist air, exercise or exertion, or emotional stress. In children, the most common triggers are viral illnesses such as those that cause the common cold (Zhao J., et. al., 2002, J Pediatr. Allergy Immunol. 13: 47-50).

Common allergens that trigger the allergic asthma include “seasonal” pollens, year-round dust mites, molds, pets, and insect parts, foods, such as fish, egg, peanuts, nuts, cow's milk, and soy, additives, such as sulfites, work-related agents, such as latex. Approximately 80% of children and 50% of adults with asthma also have allergies.

Common irritants that can trigger asthma in airways that are hyperreactive include respiratory infections, such as those caused by viral “colds,” bronchitis, and sinusitis, medication drugs, such as aspirin, other NSAIDs (nonsteroidal antiinflammatory drugs), and beta blockers (used to treat blood pressure and other heart conditions), tobacco smoke, outdoor factors such as ozone, smog, weather changes, and diesel fumes; indoor factors such as paint, detergents, deodorants, chemicals, and perfumes; nighttime GERD (gastroesophageal reflux disorder); exercise, especially under cold dry conditions; work-related factors such as chemicals, dusts, gases, and metals; emotional factors, such as laughing, crying, yelling, and distress; and hormonal factors, such as in premenstrual syndrome.

Regardless of the trigger, asthma is associated with reversible airway obstruction and airway hyperreactivity (AHR), an increased sensitivity of the airways to nonspecific stimuli such as cold air or respiratory irritants, and can be quantitated by responsiveness to methacholine or histamine. A patient diagnosed as asthmatic will generally have multiple indications over time, including wheezing, asthmatic attacks, and a positive response to methacholine challenge, i.e., a PC20 on methacholine challenge of less than about 4 mg/ml. The basic diagnosis and measurement of asthma is peak flow rates and the following diagnostic criteria are used by the British Thoracic Society (Pinnock H., and Shah R, 2007, Br. Med. J. 334 (7598): 847-50): ≧20% difference on at least three days in a week for at least two weeks; ≧20% improvement of peak flow following treatment, for example: 10 minutes of inhaled β-agonist (e.g., salbutamol), six week of inhaled corticosteroid (e.g., beclometasone), and 14 days of 30 mg prednisolone; and ≧20% decrease in peak flow following exposure to a trigger (e.g., exercise). Further guidelines for diagnosis may be found, for example, in the National Asthma Education Program Expert Panel Guidelines for Diagnosis and Management of Asthma, National Institutes of Health, 1991, Pub. No. 91-3042.

As used herein, “obesity-related asthma” refers to asthma occurring in a subject who is also obese. The subject can be obese at the time the asthma develops, at the time the diagnosis is made, and/or the time a treatment is administered. In some embodiments, the obesity can cause and/or contribute to the development of asthma and/or asthma symptoms.

The compositions and methods described herein can be administered to a subject having or diagnosed as having, e.g., asthma. In some embodiments, the methods described herein comprise administering an effective amount of compositions described herein, to a subject in order to alleviate a symptom of a condition described herein, e.g., asthma. As used herein, “alleviating a symptom” of a condition is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, or injection administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of the active ingredient that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for Chi3L1 expression levels, among others. The dosage can be determined by a physician and adjusted, as necessary, to suit observed effects of the treatment.

In some embodiments, the technology described herein relates to a pharmaceutical composition comprising an inhibitor and/or agonist as described herein (e.g. a Chi3L1 inhibitor), and optionally a pharmaceutically acceptable carrier. In some embodiments, the active ingredients of the pharmaceutical composition comprise an inhibitor and/or agonist as described herein. In some embodiments, the active ingredients of the pharmaceutical composition consist essentially of an inhibitor and/or agonist as described herein. In some embodiments, the active ingredients of the pharmaceutical composition consist of an inhibitor and/or agonist as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent as described herein.

Pharmaceutical compositions comprising, e.g, an inhibitor of Chi3L1 and/or an agonist of Sirt1, can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

A composition as described herein can be administered directly to the airways of a subject in the form of an aerosol or by nebulization. For use as aerosols, a, e.g., an inhibitor of Chi3L1 and/or an agonist of Sirt1, in solution or suspension may be packaged in a pressurized aerosol container together with suitable propellants, for example, hydrocarbon propellants like propane, butane, or isobutane with conventional adjuvants. Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as the ethers. An aerosol formulation of the invention can also comprise more than one propellant. For example, an aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present invention can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components. The aerosol formulation can be packaged under pressure and can be formulated as an aerosol using solutions, suspensions, emulsions, powders and semisolid preparations.

An inhibitor of Chi3L1 and/or an agonist of Sirt1, can also be administered in a non-pressurized form such as in a nebulizer or atomizer.

An aerosol formulation can also be a dispersion or suspension. A suspension aerosol formulation may comprise a suspension of an agent or combination of agents of the instant invention, e.g., an inhibitor of Chi3L1 and/or an agonist of Sirt1, and a dispersing agent. Dispersing agents useful in the invention include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can be formulated as an emulsion. An emulsion aerosol formulation can include, for example, an alcohol such as ethanol, a surfactant, water and a propellant, as well as an agent or combination of agents, e.g., an inhibitor of Chi3L1 and/or an agonist of Sirt1. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multidose device. As is well known, any suitable gas can be used to apply pressure during the nebulization, with preferred gases to date being those which are chemically inert to the active ingredient. Exemplary gases including, but are not limited to, nitrogen, argon or helium can be used to high advantage.

In some embodiments, the active ingredient can also be administered directly to the airways in the form of a dry powder. For use as a dry powder, e.g, an inhibitor of Chi3L1 and/or an agonist of Sirt1, can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The correct dosage of the composition is delivered to the patient.

A dry powder inhaler (i.e. TURBUHALER™ (Astra AB)) is a system operable with a source of pressurized air to produce dry powder particles of a pharmaceutical composition that is compacted into a very small volume. Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of, e.g., an inhibitor of Chi3L1 and/or an agonist of Sirt1, thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the, e.g., inhibitor of Chi3L1 and/or agonist of Sirt1, can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. By way of non-limiting example, if a subject is to be treated for inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition as described herein can be administered to a patient once. In certain embodiments, an effective dose of a composition can be administered to a patient repeatedly. For systemic administration, subjects can be administered a therapeutic amount of a composition as described herein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. asthma by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition, according to the methods described herein depend upon, for example, the form of the composition, its potency, and the extent to which symptoms, markers, or indicators of a condition described herein are desired to be reduced, for example the percentage reduction desired for symptoms. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. FEV. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. asthma symptoms and/or inflammation). It is well within the ability of one skilled in the art to monitor efficacy of administration and/or treatment by measuring any one of such parameters, or any combination of parameters. Efficacy can be assessed in animal models of a condition described herein, for example treatment of asthma. When using an experimental animal model, efficacy of treatment is evidenced when a statistically significant change in a marker is observed.

In vitro and animal model assays are provided herein which allow the assessment of a given dose of an inhibitor and/or agonist as described herein. By way of non-limiting example, the effects of a dose can be assessed in an animal model, e.g. a murine model of obesity-related asthma as described in the Examples herein.

In one aspect, described herein is a kit for performing any of the assays and/or methods described herein. In some embodiments, the kit can comprise a CHI3L1-specific reagent.

A kit is any manufacture (e.g., a package or container) comprising at least one reagent, e.g., an antibody reagent(s) or nucleic acid probe, for specifically detecting, e.g., a CHI3L1 expression product or fragment thereof, the manufacture being promoted, distributed, or sold as a unit for performing the methods or assays described herein. When the kits, and methods described herein are used for diagnosis and/or treatment of obesity, asthma, and/or obesity-related asthma, the reagents (e.g., detection probes) or systems can be selected such that a positive result is obtained in at least about 20%, at least about 40%, at least about 60%, at least about 80%, at least about 90%, at least about 95%, at least about 99% or in 100% of subjects having or developing the condition.

In some embodiments, described herein is a kit for the detection of a CHI3L1 expression product in a sample, the kit comprising at least a first CHI3L1-specific reagent as described herein which specifically binds the CHI3L1 expression product, on a solid support and comprising a detectable label. The kits described herein include reagents and/or components that permit assaying the level of an expression product in a sample obtained from a subject (e.g., a biological sample obtained from a subject). The kits described herein can optionally comprise additional components useful for performing the methods and assays described herein.

A kit can further comprise devices and/or reagents for concentrating an expression product (e.g, a polypeptide) in a sample, e.g. a plasma sample. Thus, ultrafiltration devices permitting, e.g., protein concentration from plasma can also be included as a kit component.

Preferably, a diagnostic or prognostic kit for use with the methods and assays disclosed herein contains detection reagents for CHI3L1 expression products. Such detection reagents comprise in addition to CHI3L1-specific reagents, for example, buffer solutions, labels or washing liquids etc. Furthermore, the kit can comprise an amount of a known nucleic acid and/or polypeptide, which can be used for a calibration of the kit or as an internal control. A diagnostic kit for the detection of an expression product can also comprise accessory ingredients like secondary affinity ligands, e.g., secondary antibodies, detection dyes and any other suitable compound or liquid necessary for the performance of an expression product detection method known to the person skilled in the art. Such ingredients are known to the person skilled in the art and may vary depending on the detection method carried out. Additionally, the kit may comprise an instruction leaflet and/or may provide information as to the relevance of the obtained results.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g., asthma. A subject can be male or female.

A subject can be one who has been previously diagnosed with or identified as suffering from or having a condition in need of treatment (e.g. asthma and/or obesity) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

Inhibitors of the expression of a given gene can be an inhibitory nucleic acid. In some embodiments, the inhibitory nucleic acid is an inhibitory RNA (iRNA). As used herein, the term “iRNA” refers to any type of interfering RNA, including but are not limited to RNAi, siRNA, shRNA, endogenous microRNA and artificial microRNA. Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript, e.g., via an RNA-induced silencing complex (RISC) pathway. In one embodiment, an iRNA as described herein effects inhibition of the expression and/or activity of a target gene described herein. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular—CH₂—NH—CH₂—, —CH₂—N(CH₃)—O—CH₂-[known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O—, S—, or N-alkyl; O—, S—, or N-alkenyl; O—, S- or N-alkynyl; or O-alkyl-Co-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA as described herein involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

As used herein, a particular “polypeptide”, e.g. an Sirt1 polypeptide can include the human polypeptide (e.g., SEQ ID NO: 4); as well as homologs from other species, including but not limited to bovine, dog, cat chicken, murine, rat, porcine, ovine, turkey, horse, fish, baboon and other primates. The terms also refer to fragments or variants of the native polypeptide that maintain at least 50% of the activity or effect of the native full length polypeptide, e.g. as measured in an appropriate animal model. Conservative substitution variants that maintain the activity of wildtype polypeptides will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants can be tested for activity, for example, by administering the variant to an appropriate animal model of allograft rejection as described herein.

In some embodiments, a polypeptide, e.g., a Sirt1 polypeptide, can be a variant of a sequence described herein, e.g. a variant of a Sirt1 polypeptide comprising the amino acid sequence of SEQ ID NO: 4. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or note, has more than 100% of the activity of wildtype, e.g. 110%, 125%, 150%, 175%, 200%, 500%, 1000% or more.

One method of identifying amino acid residues which can be substituted is to align, for example, the human polypeptide to a homolog from one or more non-human species. Alignment can provide guidance regarding not only residues likely to be necessary for function but also, conversely, those residues likely to tolerate change Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g. SEQ ID NO: 4, or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

In some embodiments, a polypeptide, e.g., a Sirt1 polypeptide, administered to a subject can comprise one or more amino acid substitutions or modifications. In some embodiments, the substitutions and/or modifications can prevent or reduce proteolytic degradation and/or prolong half-life of the polypeptide in the subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide, e.g., an Sirt1 polypeptide, as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., a Sirt1polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, an Sirt1 polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. a Sirt1 polypeptide, can be modified, e.g. by addition of a moiety to one or more of the amino acids comprising the peptide. In some embodiments, a polypeptide as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG or albumin.

In some embodiments, the polypeptide administered to the subject (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. In some embodiments, a polypeptide as described herein can be chemically synthesized and mutations can be incorporated as part of the chemical synthesis process.

In some embodiments, a polypeptide, e.g., a Sirt1 polypeptide, as described herein can be formulated as a pharmaceutically acceptable prodrug. As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), which are incorporated by reference herein in their entireties.

In some embodiments, a polypeptide as described herein can be a pharmaceutically acceptable solvate. The term “solvate” refers to a peptide as described herein in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The peptides of the present invention can be synthesized by using well known methods including recombinant methods and chemical synthesis. Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Humana Press, Totawa, N.J. (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, N.Y. (1984); Kimmerlin, T. and Seebach, D. J. Pept. Res. 65:229-260 (2005); Nilsson et al., Annu. Rev. Biophys. Biomol. Struct. (2005) 34:91-118; W. C. Chan and P. D. White (Eds.) Fmoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2^{nd} Ed, Oxford University Press, Cary, N.C. (2002); and P. Lloyd-Williams, F. Albericio, and E. Giralt, Chemical Approaches to the synthesis of peptides and proteins, CRC Press, Boca Raton, Fla. (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

In some embodiments, the technology described herein relates to a nucleic acid encoding a polypeptide (e.g. a Sirt1polypeptide) as described herein. As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA or cDNA. Other suitable nucleic acid molecules are RNA, including mRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

In some embodiments, a nucleic acid encoding a polypeptide as described herein (e.g. an Sirt1 polypeptide) is comprised by a vector. In some of the aspects described herein, a nucleic acid sequence encoding a given polypeptide as described herein, or any module thereof, is operably linked to a vector. In some embodiments, a nucleic acid encoding an inhibitory nucleic acid as described herein is comprised by a vector. In some of the aspects described herein, a nucleic acid sequence encoding an inhibitory nucleic acid as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments, an inhibitor of a given polypeptide can be an antibody reagent specific for that polypeptide. As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to e.g., CHI3L1.

As used herein, “expression level” refers to the number of mRNA molecules and/or polypeptide molecules encoded by a given gene that are present in a cell or sample. Expression levels can be increased or decreased relative to a reference level.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., asthma. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1. A method of treating obesity, visceral fat accumulation, asthma or obesity-related asthma, the method comprising administering an inhibitor of Chi3L1 to a subject in need of treatment for obesity, visceral fat accumulation, asthma or obesity-related asthma.

2. The method of paragraph 1, wherein the inhibitor is targeted and/or administered to white adipose tissue and/or pulmonary tissue.

3. The method of any of paragraphs 1-2, wherein the inhibitor is an antibody, antigen-binding portion thereof, or inhibitory nucleic acid.

4. The method of any of paragraphs 3, wherein the inhibitor inhibits a Chi3L1 receptor.

5. The method of paragraph 4, wherein the Chi3L1 receptor is IL-13 receptor alpha 2 (IL-13Rα2).

6. The method of any of paragraphs 1-5, further comprising administering a Sirt1 agonist.

Some embodiments of the technology described herein can be defined according to any of the following numbered paragraphs:

1. A method of treating obesity, visceral fat accumulation, asthma or obesity-related asthma, the method comprising administering an inhibitor of Chi3L1 to a subject in need of treatment for obesity, visceral fat accumulation, asthma or obesity-related asthma.

2. A method of treating asthma, the method comprising administering an inhibitor of Chi3L1 to a subject in need of treatment for asthma.

3. The method of any of paragraphs 1-2, wherein the subject is obese or diagnosed as having obesity-related asthma.

4. The method of any of paragraphs 1-3, further comprising administering a Sirt1 inhibitor.

5. A method of treating obesity, visceral fat accumulation, asthma or obesity-related asthma in a subject in need thereof, the method comprising administering a Sirt1 agonist to a subject determined to have a decreased level of Chi3L1 expression relative to a reference level.

6. The method of any of paragraphs 1-5, wherein the inhibitor is targeted or administered to white adipose tissue.

7. The method of any of paragraphs 1-6, wherein the inhibitor is targeted or administered to pulmonary white adipose tissue.

8. The method of any of paragraphs 1-7, wherein the inhibitor is targeted or administered to white adipose tissue.

9. The method of any of paragraphs 1-8, wherein the inhibitor is an antibody, antigen-binding portion thereof, or inhibitory nucleic acid.

10. The method of any of paragraphs 1-9, wherein the inhibitor inhibits a Chi3L1 receptor.

11. The method of any of paragraphs 1-10, wherein the Chi3L1 receptor is IL-13 receptor alpha 2 (IL-13Rα2).

12. A method of treating asthma in a subject in need thereof, the method comprising administering a Sirt1 agonist to a subject who is not obese or not diagnosed as having obesity-related asthma.

13. The method of paragraph 12, wherein the subject is determined to have a decreased level of Chi3L1 expression relative to a reference level.

14. A method of treating obesity, visceral fat accumulation, asthma or obesity-related asthma in a subject in need thereof, the method comprising administering a Sirt1 inhibitor to a subject determined to have an increased level of Chi3L1 expression relative to a reference level.

15. The method of paragraph 14, wherein the subject is not administered a Chi3L11 inhibitor.

16. A method of treating asthma in a subject in need thereof, the method comprising administering a Sirt1 inhibitor to a subject who is obese or diagnosed as having obesity-related asthma.

17. The method of paragraph 16, wherein the subject is determined to have an increased level of Chi3L1 expression relative to a reference level.

18. The method of any of paragraphs 16-17, wherein the subject is not administered a Chi3L11 inhibitor.

19. The method of any of paragraphs 1-18, wherein the level of Chi3L1 is the level of serum Chi3L1.

20. The method of any of paragraphs 1-18, wherein the level of Chi3L1 is the level of sputum Chi3L1.

21. The method of any of paragraphs 1-20, wherein the Sirt1 inhibitor is selected from the group consisting of:
Sirtinol; Tenovin-6; Sirtuin inhibitor IV; (S)-35; EX-527; Sumarin Sodium; Salermide; selisistat; splitomicin; AGK2; and cambinol.

22. The method of any of paragraphs 1-20, wherein the Sirt1 agonist is selected from the group consisting of:

resveratrol; re sVida; Longevinex; SRT501; SRT1720; SRT2104; and SRT2379.

### EXAMPLES

### Example 1

Obesity, especially truncal obesity, is a risk factor for asthma incidence, prevalence and severity. Chitinase 3-like-1 (Chi3l1) is an evolutionarily conserved moiety that plays a critical role in antipathogen and Th2 responses. However, the mechanisms that underlie the association between asthma and obesity and the role(s) of Chi3l1 in fat accumulation have not been defined. As demonstrated herein, Chi3l1 is regulated by a high fat diet (HFD) and simultaneously plays an important role(s) in the pathogenesis of asthma and obesity.

The regulation of Chi3l1 by a HFD and Th2 inflammation were examined. Genetically modified mice were used to define the roles of Chi3l1 in white adipose tissue (WAT) accumulation and Th2 inflammation and blockers of Sirtuin 1 (Sirt1) to define its roles in these responses. Lastly, the human-relevance of these findings was assessed with a case-control study involving obese and lean controls and asthmatics.

These studies demonstrate that a HFD and aeroallergen challenge augment the expression of WAT and pulmonary Chi3l1. Chi3l1 also played a critical role in WAT accumulation and lung Th2 inflammation. In addition, Chi3l1 inhibited Sirt1 expression and the deficient visceral fat and Th2 responses in Chi3l1 null mice were reversed by Sirt1 inhibition. Lastly, serum and sputum Chi3l1 were positively associated with truncal adiposity and serum Chi3l1 was associated with persistent asthma and low lung function in obese asthmatics.

Chi3l1 is induced by a HFD and Th2 inflammation and simultaneously contributes to the genesis of obesity and asthma.

The world is currently experiencing contemporaneous epidemics of obesity and asthma. In the U.S.A., the prevalence of obesity (defined as a body mass index ≦30 kg m⁻²) increased from approximately 15% in 1975 to 35% in 2010 (1). Epidemiologic studies have consistently demonstrated that obesity is a risk factor for asthma incidence (2, 3) and prevalence (4), as well as increased asthma severity and poor disease control (1, 5, 6). In many of these studies, the most impressive associations were between asthma and measures of abdominal or visceral obesity (1, 5, 6). Overall, the association between obesity and asthma is so striking that obese subjects with asthma are now considered to represent a ‘unique’ asthma phenotype that does not respond well to inhaled corticosteroids (4, 7, 8). The mechanistic basis for the obesity-asthma association however, has not been adequately defined (9-12). Specifically, while past studies have hypothesized that the mechanical and adipokine-related effects of visceral adiposity cause or exacerbate asthma (4, 13), the alternative possibility, that asthma and obesity share a common pathogenetic mechanism(s) that is activated in these patients, has not been addressed.

Chitinase 3-like-1 (Chi3l1) (a chitinase-like protein (CLPs), also called as YKL-40 in humans and BRP-39 in rodents) is a member of the 18 glycosyl hydrolase (GH 18) gene family, which binds to but does not degrade chitin (14).

Sirtuin 1 (Sirt1) is a histone deacetylase, that regulates a number of transcription factors that are essential for metabolism, endocrine signaling, and inflammation, including peroxisome proliferator-activated receptor γ (PPAR-γ), forkhead transcription factor, p53, and nuclear factor κB (NF-κB) (24, 25). Sirt1 is believed to be an important regulator of the energy metabolism associated with obesity and allergen-induced airway inflammation and airway hyperresponsiveness (26-28). However, its roles in these disorders have not been adequately defined and its relationship to Chi3l1 has not been addressed.

As described herein, Chi3l1 simultaneously plays a causal role in asthma-like Th2 inflammation and visceral fat accumulation. The expression and roles of Chi3l1 in animal models of asthma and obesity was characterized using wild type and Chi3l1 null mutant mice. These studies demonstrate that Chi3l1 is readily detected and significantly induced in the lung and visceral adipose tissue after allergen-challenge and/or or a high fat diet (HFD) respectively. They also demonstrate that, in the absence of Chi3l1, allergen-induced Th2 inflammation and visceral fat accumulation are significantly reduced compared to wild type controls and that these effects are mediated by Sirt1. Lastly, these murine findings were confirmed in humans where serum and sputum Chi3l1/YKL-40 correlated with parameters of truncal obesity and serum Chi3l1 was associated with persistent asthma and low lung function in obese asthmatics.

Methods

Mice Used for the Experiments.

Chi3l1/BRP-39 null mutant mice (Chi3l1^{÷}) mice were generated and characterized as previously described (20). All murine procedures were approved by the Institutional Animal Care and Use Committee at Yale University.

Obesity Studies.

Chi3l1^{-/—} male mice and wild type (WT) controls were fed regular chow (RC) or a HFD (TD88137; Harlan Inc. South Easton, Mass.) ad libitum for 12-24 weeks.

Adaptive Th2 Inflammation.

Mice were sensitized and challenged with ovalbumin (OVA) as previously described (20). One day after last challenge, the mice were sacrificed, bronchoalveolar lavage (BAL) was undertaken, and tissue responses were evaluated.

Administration of Sirtinol.

Sirtinol, a Sirt1 inhibitor (Enzo Life Sciences) or vehicle control (0.05% dimethyl sulofoxide) were diluted with Phosphate Buffered Saline (PBS) and administered via an intraperitoneal (i.p.) route.

Adipocyte Isolation.

Epididymal fat pads from Chi3l1^{-/-} and WT controls was removed and adipocytes were isolated as previously described (29-31).

Human Studies.

A case-control study design was used. 180 subjects were included. In this cohort 93 were controls and 87 subjects had asthma. Asthma was defined by a ‘provider diagnosis’ and a ‘positive’ (PC₂₀ of ≦16 mg/mL) methacholine challenge test. The methacholine challenge test was performed as per the ATS guidelines (32, 33).

Statistical Analysis.

Spearman correlations and regression analyses were mainly used in human studies. Mouse data are expressed as mean±SEM. A p value of ≦0.05 was considered to be significant.

Results

Regulation of Murine Adipose Tissue and Pulmonary Chi3l1 by a High Fat Diet.

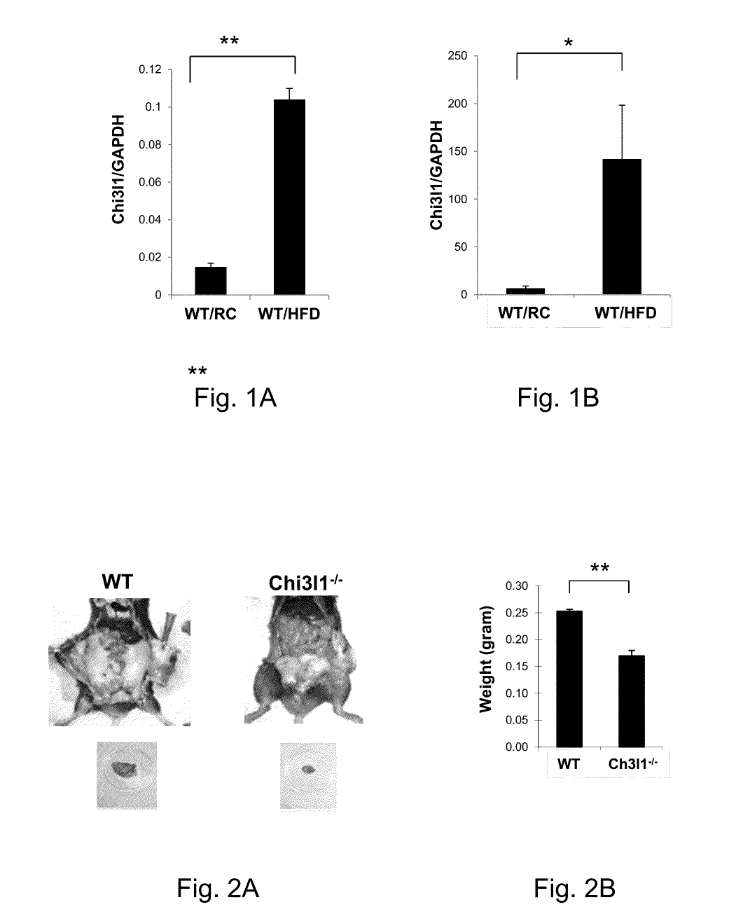

In these experiments, the expression of Chi3l1 in white adipose tissue (WAT) and pulmonary tissues was compared in mice on regular chow (RC) and a high fat diet (HFD). In mice on regular chow (RC), Chi3l1 gene expression was readily appreciated in WAT and lung tissues (FIGS. 1A and 1B). After 12-24 weeks on a HFD, a significant increase in the expression of Chi3l1 was seen in WAT and pulmonary tissues (FIGS. 1A and 1B). These studies demonstrate that Chi3l1 is expressed by WAT and lung tissues and that this expression is significantly enhanced by a HFD in both tissue compartments.

Chi3l1 Plays a Critical Role in Murine Visceral Fat Accumulation.

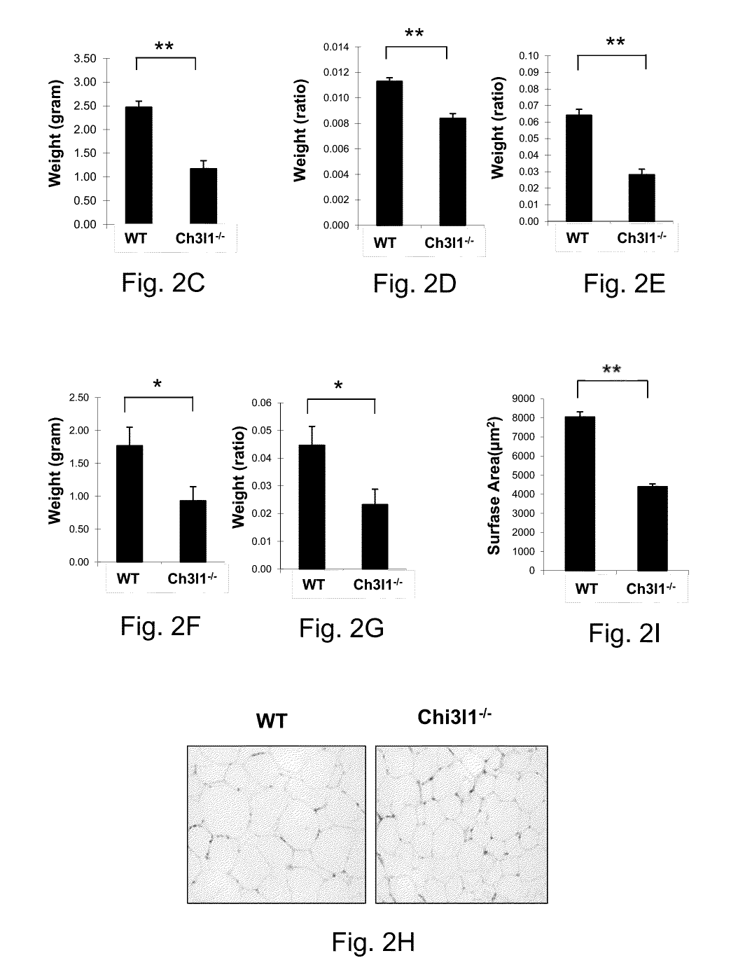

To define the role(s) of Chi3l1 in these tissues, the levels of visceral fat in Chi3l1 null mice and WT controls were compared. As can be seen in FIG. 2, abdominal visceral fat accumulation was diminished in Chi3l1 null mice compared to WT controls (FIG. 2A). In accord with these findings, the epididymal fat pads from Chi3l1 null mice were also smaller than those from controls (FIG. 2A). Since reduced visceral fat mass was seen in Chi3l1 null mice, both on regular chow and on high fat diet (FIGS. 2B and 2C, respectively), these alterations were not the result of diet. These alterations were also not related to differences in the size of the animals because the differences remained significant when total body weight was accounted for (FIGS. 2D and 2E). The alterations were also not limited to epididymal fat pads because peri-renal fat mass size (FIGS. 2F and 2G) was similarly altered. Interestingly, the reduction in visceral fat mass in Chi3l1 null mice was due, at least in part, to significantly smaller adipocyte size in Chi3l1 null mice vs. controls (FIGS. 2H and 21). These studies demonstrate that Chi3l1 stimulates visceral fat accumulation in mice.

Chi3l1 Augments Murine Visceral Fat Cytokine Elaboration.

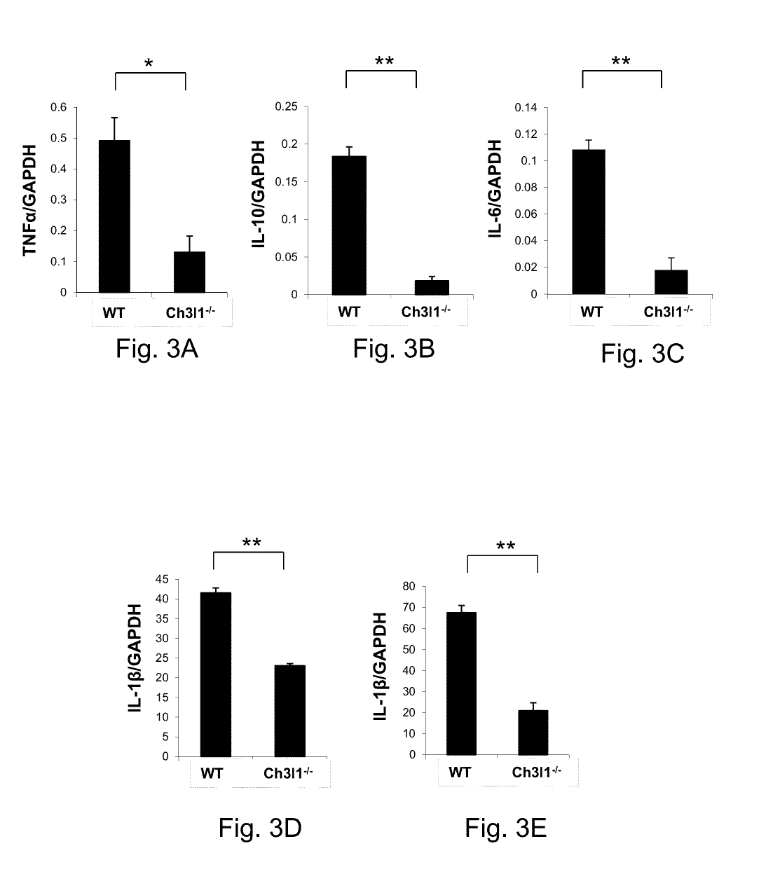

To determine if Chi3l1 regulates visceral fat cytokine expression and elaboration, the cytokine production in visceral WAT between Chi3l1 null mice and WT controls was compared. When compared to equal amounts of WAT from WT mice, the levels of mRNA encoding TNF-α, IL-10 and IL-6 were significantly lower in WAT from Chi3l1 null mice (FIGS. 3A, 3B and 3C). In accord with these findings with whole adipose tissue, adipocytes isolated from Chi3l1 null mice also produced significantly less IL-1β compared to cells from controls (FIGS. 3D and 3E). These studies demonstrate that Chi3l1 augments the expression of TNF-α, IL-6, IL-10 and IL-1β in visceral fat.

Chi3l1 Plays an Essential Role in Murine Pulmonary Th2 Inflammation.

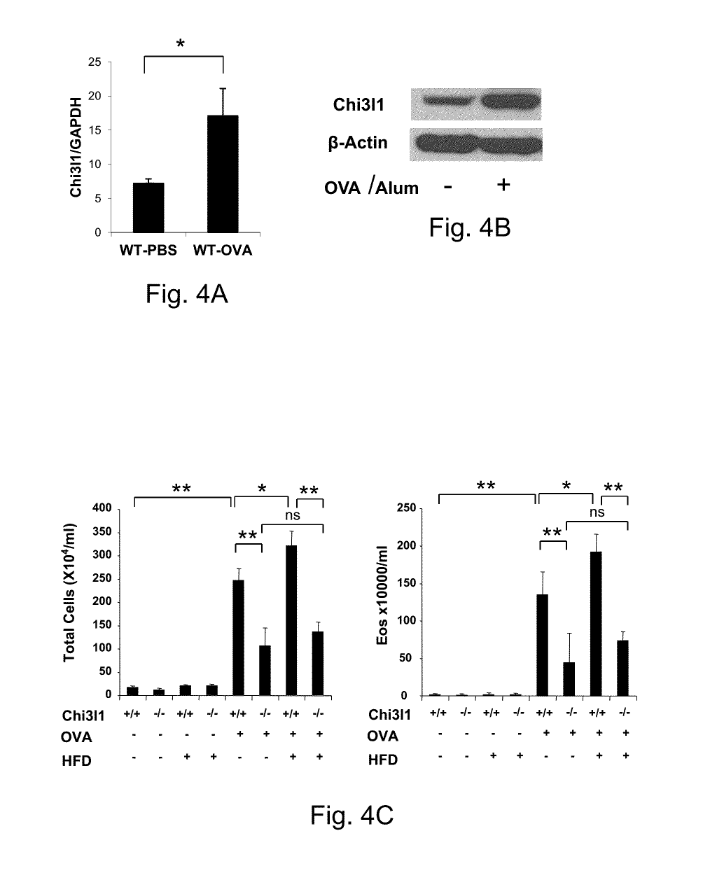

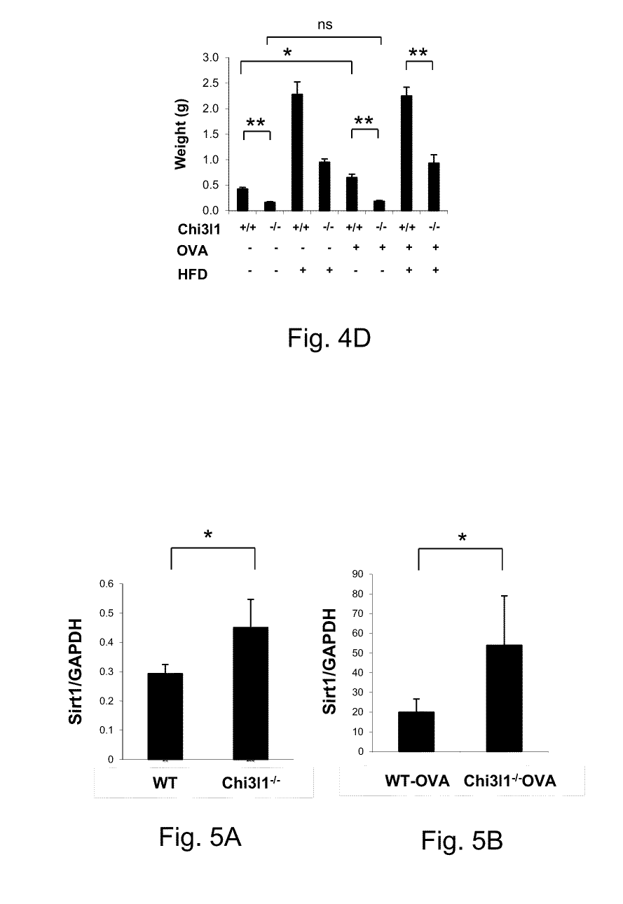

Because visceral fat mass correlates with asthma severity (34), studies were undertaken to define the role of Chi3l1 in the regulation of adaptive Th2 inflammation in the lung. Aeroallergen sensitization and challenge stimulated the expression and production of Chi3l1 (FIGS. 4A and 4B). In keeping with prior observations (20) with ovalbumin and house dust mite antigens, pulmonary aeroallergen-induced Th2 inflammation was significantly decreased in Chi3l1 null mice vs. WT controls (FIG. 4C). When viewed in combination, these studies demonstrate that Chi3l1 plays a critical role in adaptive Th2 pulmonary inflammation.

Chi3l1 Plays a Critical Role in the Allergic Inflammation and Epididymal Fat Accumulation in Mice on a HFD.

To further define the role(s) of Chi3l1 as a molecule that links obesity and asthma, we evaluated the aeroallergen-induced airway inflammatory responses and visceral fat accumulation in WT and Chi3l1^{-/-} mice on a regular diet or a HFD. These studies demonstrated that HFD-fed mice manifest a significant increase in OVA-induced BAL total cell and eosinophil recovery compared to regular diet-fed mice (FIG. 4C). In contrast, the OVA-induced responses in mice on a regular diet or a HFD were both ameliorated to comparable levels in Chi3l1 null mice (FIG. 4C). Similarly, OVA allergen sensitization and challenge significantly enhanced epididymal fat accumulation in WT mice on a regular diet and these responses were augmented in mice on high fat chow (FIG. 4D). Importantly, the OVA-induced fat responses in mice on both diets were significantly decreased in mice that lacked Chi3l1 (FIG. 4D). These studies demonstrate that Chi3l1 plays a critical role in the augmented adaptive Th2 inflammation and fat accumulation in mice on regular chow and a HFD.

Chi3l1 Inhibits Sirt1 in Murine Lung and Adipose Tissue.

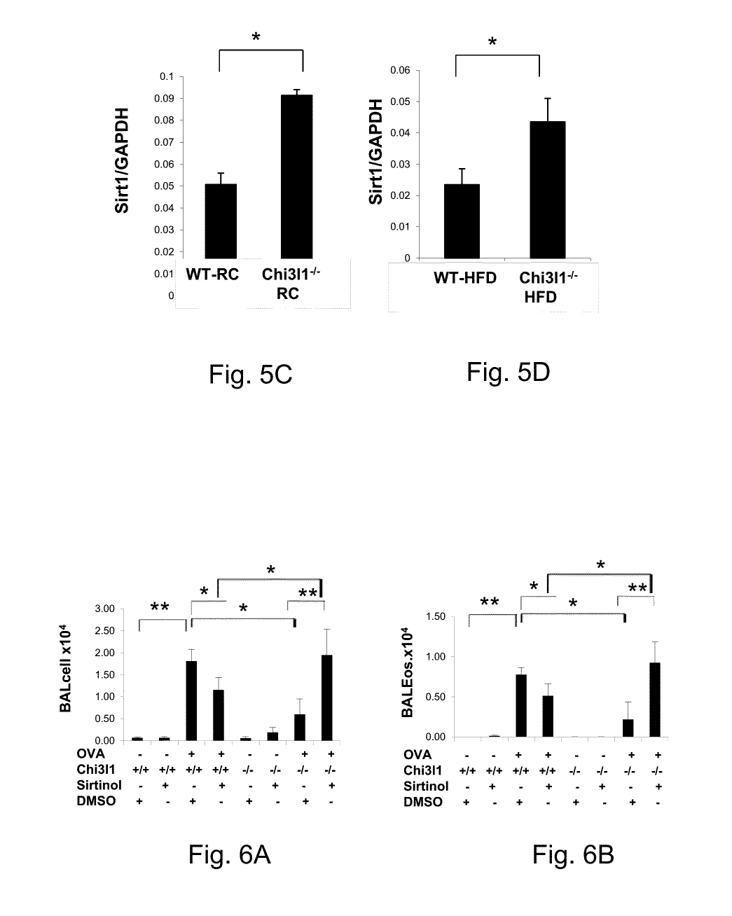

Studies using WT and genetically modified mice revealed an interesting relationship between Chi3l1 and Sirt1. Specifically, mRNA encoding Sirt1 was readily appreciated in lungs and WAT from WT mice (FIGS. 5A and 5B). In contrast, enhanced levels of pulmonary Sirt1 gene expression were seen in these tissues from Chi3l1 null mice. This difference was exaggerated after allergen induced sensitization and challenge (FIG. 5B). Similarly, in visceral WAT, the levels of Sirt1 were increased in Chi3l1 null mice when compared to controls on either regular chow or a high fat diet (FIGS. 5C and 5D). These studies demonstrate that Chi3l1 inhibits Sirt1 in both lung tissues and visceral fat.

Sirt1 Interacts with Chi3l1 to Mediate Proinflammatory and Anti-Inflammatory Effects on Murine Adaptive Th2 Inflammation.

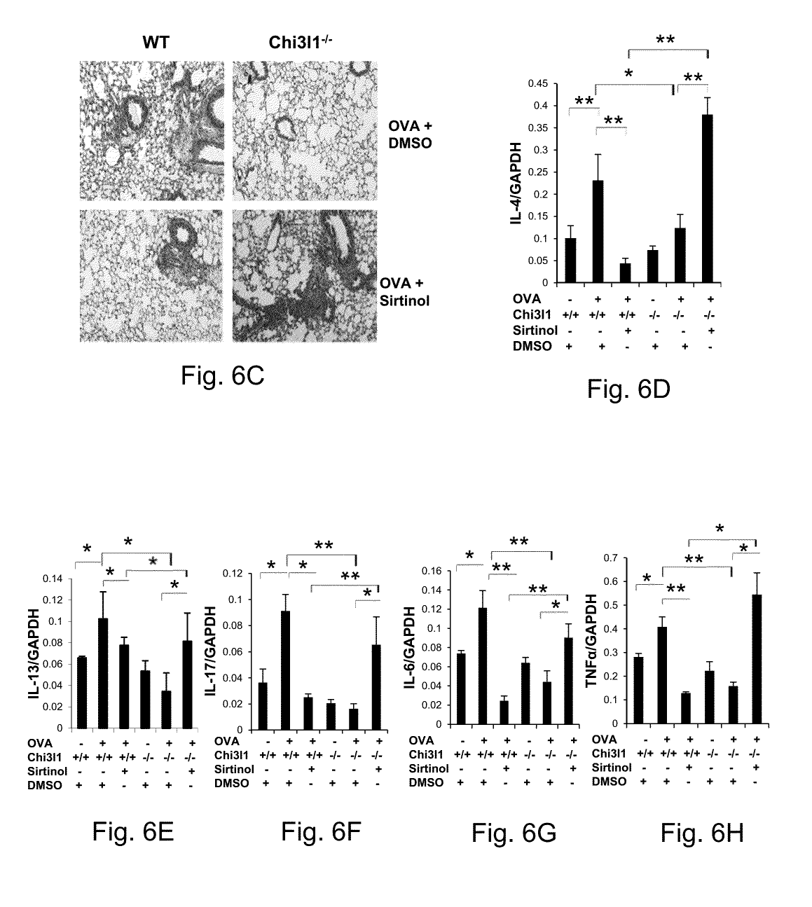

Studies were next undertaken to determine if Sirt1 played important roles in the Th2 and related responses in lungs from WT and Chi3l1 null mice. As noted above, Sirt1 gene expression was readily appreciated in lungs from WT mice and augmented in the setting of Th2 inflammation. Interestingly, systemic Sirt1 blockade in WT mice diminished aeroallergen-induced Th2 inflammation and Th2, IL-17 and IL-6 cytokine production and mucus production in the lung (FIGS. 6A-6H). As also noted above, when compared to WT controls, the levels of Th2 inflammation were markedly decreased while Sirt1 expression was enhanced in Chi3l1 null mice. In these mice, systemic Sirt1 blockade restored aeroallergen induced Th2 inflammation and Th2, IL-17 and IL-6 cytokine expression, and airway mucus to levels that approached those in WT mice (FIGS. 6A-6H). In keeping with reports in the literature, these studies demonstrate that Sirt1 has both proinflammatory and anti-inflammatory effects on pulmonary Th2 inflammation and that the effect that is seen is regulated by Chi3l1, Specifically, they demonstrate that Sirt1 augments Th2 inflammation in the presence of Chi3l1 and inhibits Th2 inflammation in the absence of Chi3l1.

Sirt1 Regulation of Murine Visceral Adipose Tissue.

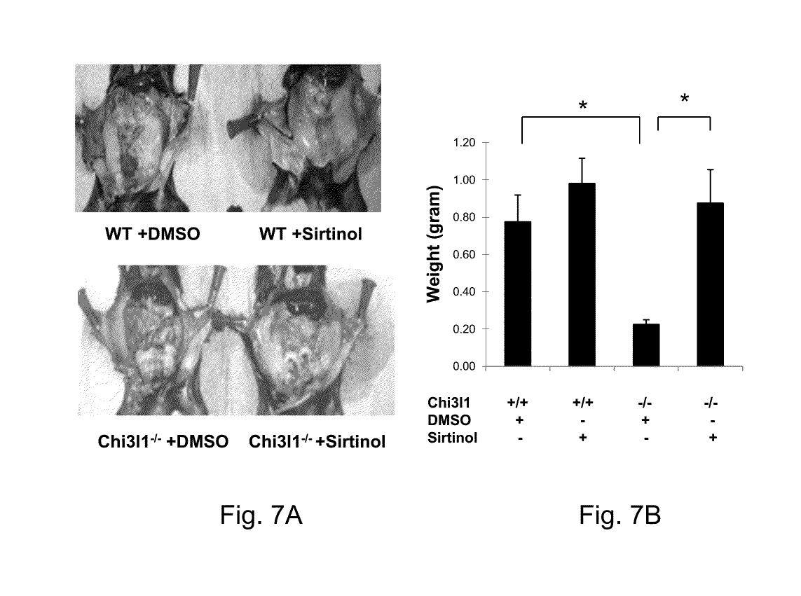

The studies noted above demonstrate that visceral fat accumulation is markedly diminished while Sirt1 expression is increased in mice that lack Chi3l1. To define the role(s) of Sirt1 in these phenotypes systemic Sirt1 blockade was employed. As can be seen in FIG. 7, Sirt1 blockade did not significantly alter the levels of visceral fat in WT mice. In contrast, Sirt1 blockade restored the levels of visceral fat in Chi3l1 null mice to levels that approached those in WT animals (FIGS. 7A and 7B). These studies demonstrate that Sirt1 inhibits visceral fat accumulation in mice that lack Chi3l1.

Serum and Sputum Chi3l1/YKL-40 are Positively and Independently Associated with Truncal Adiposity in Humans.

Studies were next undertaken to define the human relevance of the murine findings. This was first undertaken by characterizing the levels of serum Chi3l1/YKL-40 and anthropometric and DEXA parameters in patients that were lean or obese with or without asthma. These studies demonstrated that serum levels of Chi3l1/YKL-40 were positively associated with generally all mass measures, both overall (Table 1) as well as within the four individual groups (asthma or controls; lean or obese). Among all anthropometric measures, waist circumference was most strongly positively associated with serum Chi3l1/YKL-40 in the overall population, by a stepwise regression analysis (Table 1). After adjustment for standard covariates and asthma status, waist circumference was still significantly associated with serum Chi3l1/YKL-40 (Table 1). When all DEXA mass measures and BMI were instead included in a stepwise regression, the resulting statistical model included truncal lean mass, peripheral lean mass, and arm fat mass indices as the significant predictors (p≦0.006; Table 2). After adjustment for standard covariates, asthma status, and the other two DEXA mass indices, truncal lean mass was still positively associated with serum Chi3l1/YKL-40 in the overall population (p<0.001, Table 2). These studies highlight associations between Chi3l1/YKL-40 and truncal obesity in non-asthmatics and asthmatics. They are in accord with a prior study that showed that truncal lean mass (possibly measuring intravisceral fat) is the strongest obesity measure predicting asthma in women (35).

To further understand the relationships between Chi3l1/YKL-40 and obesity, the sputum levels of Chi3l1/YKL-40 were measured and used to characterize their relationship to anthropometric and DEXA parameters in the cohort. These studies demonstrated that the sputum levels of Chi3l1/YKL-40 were positively associated with generally all mass measures, both overall as well as within the four populations. Among all anthropometric measures, waist circumference was most strongly positively associated with sputum Chi3l1/YKL-40 in the overall population, by a stepwise regression analysis. Importantly, serum and sputum levels of Chi3l1/YKL-40 were separately correlated with parameters of truncal obesity and these associations were independent of each another. Specifically, when the sputum levels were accounted for, serum levels of Chi3l1/YKL-40 were still correlated with parameters of obesity and vice versa (Table 1 and 2 footnotes). Thus, in keeping with the murine studies, the present human study demonstrates that the levels of serum and sputum Chi3l1/YKL-40 correlate with truncal adiposity.

Serum Chi3l1/YKL-40 is Positively Associated with Persistent Asthma in Humans.

A study was next undertaken to define the relationship between serum Chi3l1/YKL-40 and asthma. This was done by stratifying the asthmatics into patients with intermittent and persistent asthma and comparing to controls. This study revealed that serum Chi3l1/YKL-40 concentrations were positively associated with persistent asthma (data not shown), with p≦0.04 without and with adjustment for standard covariates. Interestingly, this association was not explained by sputum Chi3l1/YKL-40 since inclusion of this variable in the statistical model did not change the results.

Serum Chi3l1/YKL-40 is Inversely Associated with FEV₁, Only in Obese Subjects with Asthma.

The correlation in humans between serum Chi3l1/YKL-40 and prebronchodilator FEV₁, a measure of asthma control and severity was examined. When analyzed as four groups (obese asthma, normal-weight asthma, obese controls, normal-weight controls, in Table 3), negative correlations were found between serum Chi3l1/YKL-40 concentrations and FEV₁ percent predicted values in the obese asthma group only (p=−0.34; p=0.01; Table 3). Thus, serum Chi3l1/YKL-40 concentrations were inversely associated with FEV₁ only when obesity and asthma were both present but not in obesity without asthma or in asthma without obesity (p value for three-way multiplicative interaction between asthma, obesity, and serum Chi3l1/YKL-40 on FEV₁ is 0.03 after adjustment for standard covariates). Finally, the association between FEV₁ and serum Chi3l1/YKL-40 in obese asthmatics was not explained away by sputum Chi3l1/YKL-40 (Table 3, footnote).

Associations of Sputum Chi3l1/YKL-40 with Asthma Outcomes do not Mirror Those of Serum Chi3l1/YKL-40.

Unlike serum Chi3l1/YKL-40, sputum Chi3l1/YKL-40 is neither associated with asthma status (data not shown) nor with percent predicted FEV₁ (data not shown). Further, unlike serum Chi3l1/YKL-40, sputum Chi3l1/YKL-40 is associated with greater systemic lipid oxidant stress (measured by urinary 8-isoprostanes), in obese asthmatics but not in normal-weight asthmatics (interaction between obese status and sputum Chi3l1/YKL-40 on urinary 8-isoprostanes among asthmatics p=0.04). These studies demonstrate that although sputum chi3l1/YKL-40 does not associate with asthma of pulmonary function it is associated with systemic oxidant stress in the obese asthma subgroup.

Discussion

Obesity has reached epidemic proportions in the USA and obesity-related illnesses have become a leading preventable cause of morbidity and even mortality (36). Asthma is one of the most prominent contributors to this morbidity and mortality since obesity augments asthma prevalence and severity and decreases responsiveness to therapy. It was hypothesized that the asthma-obesity association is due, in part, to shared regulation by Chi3l1/YKL-40. To address this hypothesis, murine modeling systems and a human case-control study were used to characterize the regulation and roles of Chi3l1 in the generation and maintenance of visceral WAT and pulmonary Th2 inflammation in mice and humans. The murine studies demonstrated that Chi3l1 is induced in visceral WAT and pulmonary tissues by a HFD and Th2 inflammation respectively. Importantly they also demonstrated that Chi3l1 plays a critical role in the pathogenesis of WAT accumulation and Th2 inflammation and that the blunted Th2 response and decrease in visceral fat accumulation that are seen in the absence of Chi3l1 are mediated, at least in part, by Sirt1. The human studies confirmed the relevance of murine studies to humans because serum and sputum Chi3l1/YKL-40 both correlated with truncal obesity; serum Chi3l1/YKL-40 was associated with persistent asthma; and serum Chi3l1/YKL-40 correlated with lower FEV₁ or worse impairment in subjects who had both obesity and asthma. When viewed in combination, these findings indicate that a high fat western diet simultaneously augments visceral adiposity and asthma by stimulating the Chi3l1 pathway.

Obesity has recently been identified as a major risk factor in the development of asthma and asthma in obese individuals tends to be more severe and refractory to treatment. However, the mechanism(s) linking obesity and asthma has not been clearly defined (2). The relationship between asthma and obesity has been assessed in a number of human studies. In many cases this association was most striking when indices of truncal obesity were employed (1, 5, 6). In accord with these observations, the murine studies demonstrated that Chi3l1 plays a critical role in asthma and obesity and the human studies revealed impressive correlations between Chi3l1/YKL-40 and truncal versus other obesity parameters. Without wishing to be bound by theory, these associations can be accounted for via a number of mechanisms. First, Chi3l1 could play critical but independent roles in the pathogenesis of asthma and obesity. Alternatively, it is possible that the effects are not independent of one another with obesity being a critical intermediary of Chi3l1 in obesity-associated asthma.

It should be noted that there are still controversies as regards the roles of eosinophils in the pathogenesis of asthma and obesity. In the former eosinophils are presumed to be detrimental and therapies are aimed at reducing their numbers or state of activation. In contrast, recent studies suggest that adipose tissue eosinophils play a physiologic role where they control and improve metabolic homeostasis (37, 40). Interestingly, in asthmatics, only the number of airway tissue eosinophils but not the number of circulating, sputum or adipose tissue eosinophils directly correlate with BMI (37,40). In addition, the number of adipose tissue eosinophils is also decreased in obese versus lean mice (38,39). When viewed in combination these studies suggest that eosinophils have different roles in different tissue compartments (40). They also raise the possibility that eosinophil redistribution plays an important role in obesity associated asthma. The present studies also provide insights into the mechanisms that may underlie these responses. By demonstrating that aeoroallergen-induced eosinphilic inflammation and WAT accumulation in mice on a HFD are abrogated in mice that lacked Chi3l1, they raise the interesting possibility that Chi3l1 is an intermediary molecule that links pulmonary Th2 and eosinophilic inflammation and allergen-stimulated visceral fat accumulation.

In prior studies, only serum Chi3l1 was evaluated and the source(s) of circulating Chi3l1 was not assessed (14, 23). The present studies evaluated the relationships between serum and sputum Chi3l1 and obesity. Interestingly, while both correlated with truncal obesity, these associations were independent of one another. In contrast, only serum Chi3l1/YKL-40 concentrations inversely associated with FEV₁ and only sputum Chi3l1 correlated with systemic lipid peroxidation in obese asthmatics. This is the first demonstration of the compartmentalization of Chi3l1 effects in humans. It indicates that Chi3l1 is regulated differently and may have different effector responses in different bodily compartments.

Sirt1 is a NAD⁺-dependent histone deacetylase that influences a diverse assortment of cellular processes through interactions with targets such as NF-Kβ, p53, FOXOs and histones as well as epigenetic programming (41). It is a nutrient sensing protein that induces hepatic gluconeogenesis, fatty acid oxidation, and adiponectin production while repressing lipogenesis and inflammation (41). However, the contribution of Sirt1 in lung inflammation is still ambiguous. Some studies suggest it has an anti-inflammatory role by inhibiting pro-inflammatory transcription factors such as NF-kβ (24). Other studies show the opposite with pharmacological inhibition of Sirt1 dampening lung Th2 inflammation by repressing PPARγ (26) or modulating vascular endothelial growth factor expression (27). The present murine studies highlight novel relationships between Chi3l1 and Sirt1 and in so doing provide at least a partial explanation for the paradox in the literature. Specifically, they demonstrate that the effect of Sirt1 blockade that is seen depends on the levels of Chi3l1 with Sirt1 exerting proinflammatory effects in the presence of Chi3l1 and anti-inflammatory effects in the absence of Chi3l1. They also demonstrate that, in the absence of Chi3l1, Sirt1 inhibits WAT accumulation. One can readily see how interventions that alter Chi3l1, its receptor(s) and or Sirt1 can be useful in efforts to control asthma, obesity, and specifically obesity-associated asthma.

Because Chi3l1 has been retained over species and evolutionary time it is assumed to play important roles in biology. To survive over evolutionary time, early cave dwellers needed to be able to mount effective antipathogen responses. In addition, regardless of the time since their last successful hunting foray, they also needed to be able to store enough fuel to be able to initiate “energetically expensive acts” such as immune activation (41). In accord with these concepts, Chi3l1 plays a critical role in antipathogen responses where it augments pathogen clearance and heightens disease tolerance (4, 22). This evolutionary conceptualization of Chi3l1 is modified herein by demonstrating that, in accord with these evolutionary principles, Chi3l1 also augments fat accumulation. However, unlike the early hunter-gatherers who lived in a world where food was scarce, modern western humans live in a world of fat-rich food abundance. Thus, one can see how the ability of Chi3l1 to augment fat/energy storage helped early humans and how it can contribute to the generation of diseases like obesity-associated asthma in modern western society.

Many of the large epidemiologic studies examining the association of obesity with asthma have noted that the effect sizes for obesity are larger in women than men (42). One explanation may be that despite its lower amount, the visceral fat in women is more metabolically active than fat from men resulting in disproportionate inflammatory effects on the lungs of women. It is also possible that female reproductive hormones regulate the Chi3l1/Sirt1 pathways. The effects of Chi3l1 are mediated by a ligand-receptor interaction with IL-13 receptor alpha 2 (IL-13Rα2) (43). Interestingly, IL-13Rα2 is encoded by a gene on chromosome X. Without wishing to be bound by theory, it is contemplated herein that Chi3l1 contributes to the gender differences in the obesity-asthma association by interacting with IL-13Rα2.

In summary, it is demonstrated herein that visceral adiposity and asthma share important Chi3l1-dependent pathways. Without wishing to be bound by theory, it is contemplated herein that a high fat Western diet simultaneously augments visceral adiposity and asthma, at least in part, via Chi3l1 stimulation. Based on this data, contemplated herein are preventive and/or therapeutic strategies for obesity-related asthma that include avoidance of fatty foods, inhibitors of Chi3l1/YKL-40 and or its receptor(s) and/or regulators of Sirt1.

### REFERENCES

1. Shore S A. Obesity and asthma: Location, location, location. Eur Respir J 2013; 41: 253-254.
2. Dixon A E, Holguin F, Sood A, Salome C M, Pratley R E, Beuther D A, Celedon J C, Shore S A. An official American Thoracic Society Workshop Report: Obesity and asthma. Proc Am Thorac Soc 2010; 7: 325-335.
3. Farah C S, Salome C M. Asthma and obesity: a known association but unknown mechanism. Respirology 2011; 17: 412-421.
4. Gruchala-Niedoszytko M, Malgorzewicz S, Niedoszytko M, Gnacinska M, Jassem E. The influence of obesity on inflammation and clinical symptoms in asthma. Advances in Medical Sciences 2013; 58: 15-21.
5. Brumpton B, Langhammer A, Romundstad P, Chen Y, Mai X M. General and abdominal obesity and incident asthma in adults: the HUNT study. Eur Respir J 2013; 41: 323-329.
6. Collins L C, Hoberty P D, Walker J F, Fletcher E C, Peiris A N. The effect of body fat distribution on pulmonary function tests. Chest 1995; 107: 1298-1302.
7. Baruwa P, Sarmah K R. Obesity and asthma. Lung India: official organ of Indian Chest Society 2013; 30: 38-46.
8. Wenzel S E. Asthma phenotypes: The evolution from clinical to molecular approaches. Nature Med 2012; 18: 716-725.
9. Grotta M B, Squebola-Cola D M, Toro A A, Ribeiro M A, Mazon S B, Ribeiro J D, Antunes E. Obesity increases eosinophil activity in asthmatic children and adolescents. BMC Pulm Med 2013; 13: 39.
10. Aydin M, Koca C, Ozol D, Uysal S, Yildirim Z, Kavakli H S, Yigitoglu M R. Interaction of Metabolic Syndrome with Asthma in Postmenopausal Women: Role of Adipokines. Inflammation 2013.
11. Ali Z, Ulrik C S. Obesity and asthma: A coincidence or a causal relationship? A systematic review. Respir Med 2013; (in press).
12. Farah C S, Salome C M. Asthma and obesity: a known association but unknown mechanism. Respirology 2012; 17: 412-421.
13. Dietze J, Bocking C, Heverhagen J T, Voelker M N, Renz H. Obesity lowers the threshold of allergic sensitization and augments airway eosinophilia in a mouse model of asthma. Allergy 2012; 67: 1519-1529.
14. Lee C G, Da Silva C A, Dela Cruz C S, Ahangari F, Ma B, Kang M J, He C H, Takyar S, Elias J A. Role of chitin and chitinase/chitinase-like proteins in inflammation, tissue remodeling, and injury. Annu Rev Physiol 2011; 73: 479-501.
15. Aerts J M, van Breemen M J, Bussink A P, Ghauharali K, Sprenger R, Boot R G, Groener J E, Hollak C E, Maas M, Smit S, Hoefsloot H C, Smilde A K, Vissers J P, de Jong S, Speijer D, de Koster C G. Biomarkers for lysosomal storage disorders: identification and application as exemplified by chitotriosidase in Gaucher disease. Acta Paediatr Suppl 2008; 97: 7-14.
16. Funkhouser J D, Aronson N N, Jr. Chitinase family GH18: evolutionary insights from the genomic history of a diverse protein family. BMC Evol Biol 2007; 7: 96.
17. Areshkov P O, Avdieiev S S, Balynska O V, Leroith D, Kaysan V M. Two closely related human members of chitinase-like family, CHI3L1 and CHI3L2, activate ERK1/2 in 293 and U373 cells but have the different influence on cell proliferation. Int J Biol Sci 2012; 8: 39-48.
18. Chen C-C, Llado V, Eurich K, Tran H T, Mizoguchi E. Carbohydrate-binding motif in chitinase 3-like 1 (CH13L1/YKL-40) specifically activates Akt signaling pathway in colonic epithelial cells. Clin Immunol 2011; 140: 268-275.
19. Kim M N, Lee K E, Hong J Y, Heo W I, Kim K W, Kim K E, Sohn M H. Involvement of the MAPK and PI3K pathways in chitinase 3-like 1-regulated hyperoxia-induced airway epithelial cell death. Biochemical and biophysical research communications 2012; 421: 790-796.
20. Lee C G, Hartl D, Lee G R, Koller B, Matsuura H, Da Silva C A, Sohn M H, Cohn L, Homer R J, Kozhich A A, Humbles A, Kearley J, Coyle A, Chupp G, Reed J, Flavell R A, Elias J A. Role of breast regression protein 39 (BRP-39)/chitinase 3-like-1 in Th2 and IL-13-induced tissue responses and apoptosis. The Journal of Experimental Medicine 2009; 206: 1149-1166.
21. Sohn M H, Kang M J, Matsuura H, Bhandari V, Chen N Y, Lee C G, Elias J A. The chitinase-like proteins breast regression protein-39 and YKL-40 regulate hyperoxia-induced acute lung injury. Am J Respir Crit Care Med 2010; 182: 918-928.
22. Dela Cruz C S, Liu W, He C H, Jacoby A, Gornitzky A, Ma B, Flavell R, Lee C G, Elias J A. Chitinase 3-like-1 promotes Streptococcus pneumoniae killing and augments host tolerance to lung antibacterial responses. Cell Host & microbe 2012; 12: 34-46.
23. Coffman F D. Chitinase 3-Like-1 (CHI3L1): a putative disease marker at the interface of proteomics and glycomics. Crit Rev Clin Lab Sci 2008; 45: 531-562.
24. Yang S R, Wright J, Bauter M, Seweryniak K, Kode A, Rahman I. Sirtuin regulates cigarette smoke-induced proinflammatory mediator release via RelA/p65 NF-kappaB in macrophages in vitro and in rat lungs in vivo: implications for chronic inflammation and aging. American Journal of Physiology Lung Cellular and Molecular Physiology 2007; 292: L567-576.
25. Yang T, Fu M, Pestell R, Sauve A A. SIRT1 and endocrine signaling. Trends in endocrinology and metabolism: TEM 2006; 17: 186-191.
26. Legutko A, Marichal T, Fievez L, Bedoret D, Mayer A, de Vries H, Klotz L, Drion P V, Heirman C, Cataldo D, Louis R, Thielemans K, Andris F, Leo O, Lekeux P, Desmet C J, Bureau F. Sirtuin 1 promotes Th2 responses and airway allergy by repressing peroxisome proliferator-activated receptor-gamma activity in dendritic cells. Journal of immunology (Baltimore, Md.: 1950) 2011; 187: 4517-4529.
27. Kim S R, Lee K S, Park S J, Min K H, Choe Y H, Moon H, Yoo W H, Chae H J, Han M K, Lee Y C. Involvement of sirtuin 1 in airway inflammation and hyperresponsiveness of allergic airway disease. The Journal of Allergy and Clinical Immunology 2010; 125: 449-460 e414.
28. Zillikens M C, van Meurs J B, Rivadeneira F, Amin N, Hofman A, Oostra B A, Sijbrands E J, Witteman J C, Pols H A, van Duijn C M, Uitterlinden A G. SIRT1 genetic variation is related to BMI and risk of obesity. Diabetes 2009; 58: 2828-2834.
29. Shaul M E, Bennett G, Strissel K J, Greenberg A S, Obin M S. Dynamic, M2-like remodeling phenotypes of CD11c+ adipose tissue macrophages during high-fat diet—induced obesity in mice. Diabetes 2010; 59: 1171-1181.
30. Vijay-Kumar M, Aitken J D, Carvalho F A, Cullender T C, Mwangi S, Srinivasan S, Sitaraman S V, Knight R, Ley R E, Gewirtz A T. Metabolic syndrome and altered gut microbiota in mice lacking Toll-like receptor 5. Science 2010; 328: 228-231.
31. Bassaganya-Riera J, Misyak S, Guri A J, Hontecillas R. PPAR gamma is highly expressed in F4/80(hi) adipose tissue macrophages and dampens adipose-tissue inflammation. Cellular Immunology 2009; 258: 138-146.
32. Crapo R O, Casaburi R, Coates A L, Enright P L, Hankinson J L, Irvin C G, Maclntyre N R, McKay R T, Wanger J S, Anderson S D, Cockcroft D W, Fish J E, Sterk P J. Guidelines for methacholine and exercise challenge testing-1999. This official statement of the American Thoracic Society was adopted by the ATS Board of Directors, July 1999. Am J Respir Crit Care Med 2000; 161: 309-329.
33. Miller M R, Hankinson J, Brusasco V, Burgos F, Casaburi R, Coates A, Crapo R, Enright P, van der Grinten C P M, Gustafsson P, Jensen R, Johnson D C, Maclntyre N, McKay R, Navajas D, Pedersen O F, Pellegrino R, Viegi G, Wanger J. Standardisation of spirometry. Eur Respir J 2005; 26: 319-338.
34. Von Behren J, Lipsett M, Horn-Ross P L, Delfino R I, Gilliland F, McConnell R, Bernstein L, Clarke C A, Reynolds P. Obesity, waist size and prevalence of current asthma in the California Teachers Study cohort. Thorax 2009; 64: 889-893.
35. Sood A, Qualls C, Li R, Schuyler M, Beckett W S, Smith L J, Thyagarajan B, Lewis C E, Jacobs D R. Lean mass predicts asthma better than fat mass among females. Eur Respir J 2011; 37: 65-71.
36. Liu C, Elmquist J K. Tipping the scales early: Probing the long-term effects of obesity. J Clin Invest 2012; 122: 3840-3842.
37. Desai D, Newby C, Symon F A, Haldar P, Shah S, Gupta S, Bafadhel M, Singapuri A, Siddiqui S, Woods J, Herath A, Anderson I K, Bradding P, Green R, Kulkarni N, Pavord I, Marshall R P, Sousa A R, May R D, Wardlaw A J, Brightling C E. Elevated sputum interleukin-5 and submucosal eosinophilia in obese individuals with severe asthma. Am J Respir Crit Care Med 2013; 188: 657-663.
38. Wu D, Molofsky A B, Liang H E, Ricardo-Gonzalez R R, Jouihan H A, Bando J K, Chawla A, Locksley R M. Eosinophils sustain adipose alternatively activated macrophages associated with glucose homeostasis. Science 2011; 332: 243-247.
39. Molofsky A B, Nussbaum J C, Liang H E, Van Dyken S J, Cheng L E, Mohapatra A, Chawla A, Locksley R M. Innate lymphoid type 2 cells sustain visceral adipose tissue eosinophils and alternatively activated macrophages. The Journal of Experimental Medicine 2013; 210: 535-549.
40. Lloyd C M, Saglani S. Eosinophils in the spotlight: Finding the link between obesity and asthma. Nature Medicine 2013; 19: 976-977.
41. Gillum M P, Kotas M E, Erion D M, Kursawe R, Chatterjee P, Nead K T, Muise E S, Hsiao J J, Frederick D W, Yonemitsu S, Banks A S, Qiang L, Bhanot S, Olefsky J M, Sears D D, Caprio S, Shulman G I. SirT1 regulates adipose tissue inflammation. Diabetes 2011; 60: 3235-3245.
42. Weiss S T, Shore S. Obesity and asthma: directions for research. Am J Respir Crit Care Med 2004; 169: 963-968.
43. He C H, Lee C G, Dela Cruz C S, Lee C M, Zhou Y, Ahangari F, Ma B, Herzog E L, Rosenberg S A, Li Y, Nour A M, Parikh C R, Schmidt I, Modis Y, Cantley L, Elias J A. Chitinase 3-like 1 Regulates Cellular and Tissue Responses via IL-13 Receptor alpha2. Cell Reports 2013; 4: 830-841.

TABLE 1
Association between serum Chi3l1/YKL-40 and
anthropometric measures in humans.

| BMI/Skinfold | Univariate analysis | | Multivariable analysis | |
|---|---|---|---|---|
| thickness measure | (n = 180) | | (n = 180)^{Note 1} | |
| variable | Standardized β | P value | Standardized β | P value |
| Waist circumference | 0.37 | <0.001 | 0.37 | <0.001 |
| Hip circumference | 0.29 | <0.001 | | |
| Waist to hip ratio | 0.31 | <0.001 | | |
| Subscapular | 0.22 | 0.003 | | |
| skinfold thickness | | | | |
| Triceps skinfold | 0.20 | 0.006 | | |
| thickness | | | | |
| Chest skinfold | 0.30 | <0.001 | | |
| thickness | | | | |
| Midaxillary skinfold | 0.32 | <0.001 | | |
| thickness | | | | |
| Abdominal skinfold | 0.27 | <0.001 | | |
| thickness | | | | |
| Suprailiac skinfold | 0.23 | 0.002 | | |
| thickness | | | | |
| Thigh skinfold | 0.18 | 0.02 | | |
| thickness | | | | |
| BMI | 0.33 | <0.001 | | |

^{Note 1}In the fully-adjusted multivariable model, asthma status and standard covariates were included.
Note 2:
Waist circumference was the strongest predictor in a stepwise regression and therefore the multivariable analysis was performed only for this measure. Additional adjustment for sputum YKL-40 concentrations in the multivariable analysis did not change the results (standardized β = 0.46, p < 0.001).

TABLE 2
Association between serum Chi3l1/YKL-40 and Dual-energy X-ray
Absorptiometry (DEXA) fat and lean mass measures in humans.

| | Univariate analysis | | Multivariable analysis | |
|---|---|---|---|---|
| BMI/DEXA mass | (n = 174) | | (n = 174)^{Note 1} | |
| measure variable | Standardized β | P value | Standardized β | P value |
| Total fat mass index | 0.27 | <0.001 | | |
| Truncal fat mass | 0.31 | <0.001 | | |
| index | | | | |
| Peripheral fat mass | 0.21 | 0.004 | | |
| index | | | | |
| Arm fat mass index | 0.29 | <0.001 | 0.27 | 0.006 |
| Leg fat mass index | 0.18 | 0.02 | | |
| Total lean mass | 0.26 | <0.001 | | |
| index | | | | |
| Truncal lean mass | 0.34 | <0.001 | 0.51 | <0.001 |
| index | | | | |
| Peripheral lean | 0.13 | 0.09 | −0.41 | 0.001 |
| mass index | | | | |
| Arm lean mass | 0.08 | 0.27 | | |
| index | | | | |
| Leg lean mass index | 0.13 | 0.08 | | |
| Body mass index | 0.33 | <0.001 | | |
| (BMI) | | | | |

^{Note 1}In the fully-adjusted multivariable model, asthma status, standard covariates, and the three DEXA mass indices were included.
Note 2:
Truncal lean mass, peripheral lean mass and arm fat mass indices were the strongest predictors in a stepwise regression and therefore the multivariable analysis was performed only for these measures. The separate relationships between truncal lean mass, peripheral lean mass, and arm fat mass indices with serum YKL-40 were generally unchanged after adding sputum YKL-40 in the multivariable model (standardized β of 0.57, −0.29, 0.22 respectively with p values of <0.001, 0.04, 0.053 respectively).
Note 3:
All indices were obtained by dividing the weight of that measure by the square of height (in kg/m²). Six of 180 eligible individuals did not have DEXA evaluations.

TABLE 3
Spearman correlations between serum Chi3l1/YKL-40 concentrations
with pre-bronchodilator FEV₁ percent predicted values.

| | Obese asthma | Normal-weight | Obese controls | Normal-weight |
|---|---|---|---|---|
| | (n = 51) | asthma (n = 34) | (n = 44) | controls (n = 48) |
| % predicted FEV₁ - | −0.34 (p = 0.01) | 0.01 (p = 0.97;) | 0.09 (p = 0.56) | 0.03 (p = 0.86) |
| unadjusted | | | | |
| % predicted FEV₁ - | −0.40 (p = 0.005) | −0.19 (p = 32) | 0.26 (p = 0.12) | 0.003 (p = 0.99) |
| adjusted for standard | | | | |
| covariates | | | | |

Note 1:
Subjects with asthma included those with intermittent disease as well as persistent disease. The above correlations appeared stronger in obese subjects with persistent asthma than obese subjects with intermittent asthma (ρ = −0.35, adjusted p = 0.049 vs. ρ = −0.03, adjusted p = 0.93; although the interaction was not significant p = 0.50).
Note 2:
There was a three-way multiplicative interaction between asthma status, obesity status, and serum YKL-40 concentrations on baseline FEV₁ percent predicted, p = 0.03 after adjustment for standard covariates.
Note 3:
When adjusted for sputum Chi3l1/YKL-40, the correlation between serum Chi3l1/YKL-40 and FEV₁ in obese asthmatic group was unchanged from the unadjusted value (ρ = −0.39, p = 0.009, n = 44). Correlations in remaining three groups remained non-significant. However, an independent association between sputum Chi3l1/YKL-40 and % predicted FEV₁ was not seen in unadjusted or adjusted analyses.

What is claimed herein is:

1. A method of treating obesity, visceral fat accumulation, asthma or obesity-related asthma, the method comprising administering an inhibitor of Chi3L1 to a subject in need of treatment for obesity, visceral fat accumulation, asthma or obesity-related asthma.

2. A method of treating asthma, the method comprising administering an inhibitor of Chi3L1 to a subject in need of treatment for asthma.

3. The method of claim 2, wherein the subject is obese or diagnosed as having obesity-related asthma.

4. The method of claim 2, wherein the inhibitor is targeted or administered to white adipose tissue.

5. The method of claim 2, wherein the inhibitor is targeted or administered to pulmonary white adipose tissue.

6. The method of claim 2, wherein the inhibitor is targeted or administered to white adipose tissue.

7. The method of claim 2, wherein the inhibitor is an antibody, antigen-binding portion thereof, or inhibitory nucleic acid.

8. The method of claim 2, wherein the inhibitor inhibits a Chi3L1 receptor.

9. The method of claim 8, wherein the Chi3L1 receptor is IL-13 receptor alpha 2 (IL-13Rα2).

10. The method of claim 2, further comprising administering a Sirt1 inhibitor.

11. The method of claim 2, wherein the level of Chi3L1 is the level of serum Chi3L1.

12. The method of claim 2, wherein the level of Chi3L1 is the level of sputum Chi3L1.

13. A method of treating asthma in a subject in need thereof, the method comprising administering a Sirt1 inhibitor to a subject who is obese or diagnosed as having obesity-related asthma.

14. The method of claim 13, wherein the subject is determined to have an increased level of Chi3L1 expression relative to a reference level.

15. The method of claim 13, wherein the subject is not administered a Chi3L11 inhibitor.
